(19) United States
(12) Patent Application Publication
(10) Pub. No.: US 2025/0268551 A1
(43) Pub. Date: Aug. 28, 2025

(54) MEDICAL SYSTEM AND RECORDING MEDIUM

(71) Applicant: GE Precision Healthcare LLC, Waukesha, WI (US)

(72) Inventors: Ryosuke Fujimoto, Hino (JP); Kentaro Ogata, Hino (JP)

(21) Appl. No.: 19/065,932

(22) Filed: Feb. 27, 2025

(30) Foreign Application Priority Data
Feb. 27, 2024 (JP) 2024-027948

(51) Int. Cl. A61B 6/00; A61B 6/03; A61B 6/40; A61B 6/58

(52) U.S. Cl. CPC A61B 6/54; A61B 6/03; A61B 6/4035; A61B 6/585

(57) ABSTRACT

Described is a medical system and method for setting a first delay time from when the tube voltage is switched to a high kV to when data collection starts and a second delay time from when the tube voltage is switched to a low kV to when data collection starts, determining a high-kV first effective voltage value when a high kV is applied to the X-ray tube 104, determining a low-kV second effective voltage value when a low kV is applied to the X-ray tube 104, and calculating an effective voltage difference representing the difference between the effective voltage value and the effective voltage value. Furthermore, to execute the abovementioned steps, a plurality of times to acquire a plurality of effective voltage differences, and execute obtaining of a first optimal delay time and the second optimal delay time based on the plurality of effective voltage differences.

### CROSS-REFERENCE TO RELATED APPLICATIONS

This application claims priority to Japanese Application No 2024-027948, filed on Feb. 27, 2024, the disclosure of which is incorporated herein by reference in its entirety.

### TECHNICAL FIELD

The present invention relates to a medical system capable of switching tube voltages, and a recording medium in which an instruction for controlling the medical system is recorded.

### BACKGROUND ART

A CT system is known as a medical device that noninvasively images a subject. CT systems can acquire tomographic images of a subject in a short scanning time and therefore are widely used in hospitals and other medical facilities.

The CT system applies a prescribed voltage to a cathode-anode tube of an X-ray tube to generate X-rays. The generated X-rays penetrate the subject and are detected by a detector. The CT system reconstructs a CT image of the subject based on data detected by the detector.

### SUMMARY OF THE INVENTION

Single energy CT (SECT) is a well-known imaging technique for CT systems. SECT is a method for obtaining a CT image of a subject by applying a prescribed voltage (e.g., 120 kV) to a cathode-anode tube of an X-ray tube to generate X-rays. However, in SECT, CT values may be close even for different substances, and identification of different substances may be difficult.

Therefore, DECT (Dual Energy CT) technology is being researched and developed. DECT is a technology that uses X-rays in different energy regions to distinguish between substances, and can acquire images that are useful for diagnosis in clinical settings, and thus is beginning to come into widespread use. In the DECT technology, a kV switching technology is known that switches the tube voltage of the X-ray tube between a low tube voltage and a high tube voltage.

In the kV switching technology, data acquired by the detector must be separated into data corresponding to a high tube voltage and data corresponding to a low tube voltage. In general, if the voltage difference between the high tube voltage and the low tube voltage is small, the accuracy of identifying substances (accuracy of identifying data) tends to be low. Therefore, in the kV switching technology, the tube voltage is set such that the difference between the high tube voltage and the low tube voltage is as large as possible.

On the other hand, in kV switching, it is necessary to switch between a low tube voltage and a high tube voltage in a short period of time. Ideally, when the tube voltage is switched, the voltage is preferably changed to a rectangular shape. However, in reality, when the tube voltage is switched, a certain amount of time is required for the tube voltage to reach a desired value. For example, when the tube voltage is switched from a low tube voltage to a high tube voltage, a certain rise time is required for the tube voltage to reach a desired value. Similarly, when the tube voltage is switched from a high tube voltage to a low tube voltage, a certain fall time is required for the tube voltage to reach a desired value. Therefore, in order to reduce the effects of the rise time and fall time of the tube voltage, the CT system does not start collecting data at the same time as switching the tube voltage, but instead provides a delay time between switching the tube voltage and starting to collect data for the corresponding tube voltage.

However, there are individual differences in the rise time and fall time of the tube voltage of CT systems, and the delay time set in one CT system is not necessarily an appropriate delay time for another CT system. Furthermore, when the CT system is used for a long period of time, parts deteriorate over time, and as a result, the delay time currently being used deviates from the ideal delay time, resulting in a problem of deterioration in image quality. Therefore, a technology capable of obtaining an optimal delay time is desired. A first aspect of the present disclosure is a medical system including an X-ray tube configured to be able to switch a tube voltage applied to the X-ray tube between a first tube voltage and a second tube voltage, a detector assembly for detecting X-rays irradiated from the X-ray tube, the detector assembly including a reference region for detecting X-rays and a filter, the reference region including a first sub-region for detecting X-rays that have passed through the filter and a second sub-region for detecting X-rays that have not passed through the filter, and one or a plurality of processors for executing the following: setting a first delay time from switching the tube voltage to the first tube voltage to starting data collection, and a second delay time from switching the tube voltage to the second tube voltage to starting data collection; determining a first effective voltage value of the first tube voltage based on energy information of X-rays detected in the first sub-region of the detector assembly and energy information of X-rays detected in the second sub-region of the detector assembly, when the first tube voltage is applied to the X-ray tube; determining a second effective voltage value of the second tube voltage based on energy information of X-rays detected in the first sub-region of the detector assembly and energy information of X-rays detected in the second sub-region of the detector assembly, when the second tube voltage is applied to the X-ray tube; and calculating an effective voltage difference representing the difference between the first effective voltage value and the second effective voltage value, wherein the one or more processors execute, a plurality of times, setting the first delay time and the second delay time, determining the first effective voltage value, determining the second effective voltage value, and calculating the effective voltage difference, to acquire a plurality of effective voltage differences, and obtain the first delay time and the second delay time when a maximum effective voltage difference is obtained based on the plurality of effective voltage differences.

Furthermore, a second aspect of the present invention is a non-transitory storing medium in which is stored an instruction executed by one or a plurality of processors of a medical system including an X-ray tube configured to be able to switch a tube voltage applied to the X-ray tube between a first tube voltage and a second tube voltage, a detector assembly for detecting X-rays irradiated from the X-ray tube, the detector assembly including a reference region for detecting X-rays and a filter, the reference region including a first sub-region for detecting X-rays that have passed through the filter and a second sub-region for detecting X-rays that have not passed through the filter, and one or a plurality of processors, wherein the instruction, upon being executed by the one or plurality of processors, causes the one or plurality of processors to execute the following: setting a first delay time from switching the tube voltage to the first tube voltage to starting data collection, and a second delay time from switching the tube voltage to the second tube voltage to starting data collection; determining a first effective voltage value of the first tube voltage based on energy information of X-rays detected in the first sub-region of the detector assembly and energy information of X-rays detected in the second sub-region of the detector assembly, when the first tube voltage is applied to the X-ray tube; determining a second effective voltage value of the second tube voltage based on energy information of X-rays detected in the first sub-region of the detector assembly and energy information of X-rays detected in the second sub-region of the detector assembly, when the second tube voltage is applied to the X-ray tube; and calculating an effective voltage difference representing the difference between the first effective voltage value and the second effective voltage value, and the instruction, upon being executed by the one or plurality of processors, causes the one or plurality of processors to further execute, a plurality of times, setting the first delay time and the second delay time, determining the first effective voltage value, determining the second effective voltage value, and calculating the effective voltage difference, to acquire a plurality of effective voltage differences, and obtain the first delay time and the second delay time when a maximum effective voltage difference is obtained based on the plurality of effective voltage differences.

The present invention acquires a plurality of effective voltage differences by executing, a plurality of times, setting the first delay time and the second delay time, determining the first effective voltage value, determining the second effective voltage value, and calculating the effective voltage difference. Therefore, a delay time suitable for setting the effective voltage difference to a large value can be obtained based on a plurality of effective voltage differences.

### BRIEF DESCRIPTION OF THE DRAWINGS

FIG. 1 is a block diagram of a CT system 100 of the present embodiment;

FIG. 2 is an explanatory diagram when switching a tube voltage;

FIG. 3 is an explanatory diagram of a high kV period and a low kV period of a tube voltage waveform;

FIG. 4 is a diagram depicting an example of an effective voltage value VH corresponding to a tube voltage in a period 21 and an effective voltage value VL corresponding to a tube voltage in a period 22;

FIG. 5 is an explanatory diagram of a delay time;

FIG. 6 is a diagram depicting an effective voltage difference obtained for a rectangular waveform 30;

FIG. 7 is an explanatory diagram of a detector assembly 108;

FIG. 8 is an enlarged view of a reference region 109 of the detector assembly 108;

FIG. 9 is a flowchart of a method for creating a baseline;

FIG. 10 is diagram depicting a method for calculating an index value M;

FIG. 11 is an explanatory diagram of a method for creating a baseline;

FIG. 12 is a diagram depicting a baseline 60;

FIG. 13 is a flowchart for obtaining an optimal delay time during installation work of the CT system;

FIG. 14 is an explanatory diagram of a reference delay time;

FIG. 15 is an explanatory diagram of a method for calculating an index value M (=MHr) corresponding to high kV and an index value M (=MLr) corresponding to low kV;

FIG. 16 is an explanatory diagram of a method for determining an effective voltage value;

FIG. 17 is a specific explanatory diagram of a method for determining effective voltage values VHr and VLr using a baseline 60;

FIG. 18 is a diagram depicting an example in which a delay time is set to a time longer than a reference delay time;

FIG. 19 is an explanatory diagram of a method for calculating an index value M (=MHL) corresponding to high kV and an index value M (=MLL) corresponding to low kV;

FIG. 20 is an explanatory diagram of a method for determining an effective voltage value;

FIG. 21 is a diagram depicting an example in which a delay time is set to a time shorter than a reference delay time;

FIG. 22 is an explanatory diagram of a method for calculating an index value M (=MHS) corresponding to high kV and an index value M (=MLS) corresponding to low kV;

FIG. 23 is a diagram depicting data sets Q1, Q2, and Q3;

FIG. 24 is a flowchart of a method for obtaining an optimal delay time when a user periodically executes calibration of the CT system; FIG. 25 is an explanatory diagram of a latest value of a registered delay time;

FIG. 26 is a diagram depicting a delay time set to a time different from a registered delay time (TAN, TBN);

FIG. 27 is an explanatory diagram of a method for calculating a fifth effective voltage difference D5; and

FIG. 28 is a diagram depicting data sets Q4 and Q5.

### DETAILED DESCRIPTION OF THE DRAWINGS

An embodiment for carrying out the invention will be described below, but the present invention is not limited to the following embodiment.

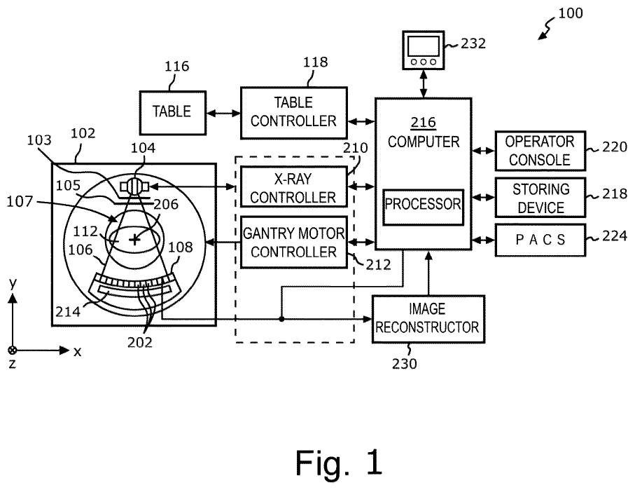

FIG. 1 is a block diagram of a CT system 100 of the present embodiment. The CT system 100 includes a gantry 102 and a table 116. The gantry 102 includes a bore 107. A subject 112 is transported through the bore 107 and then the subject 112 is scanned. An X-ray tube 104, a filter part 103, a pre-collimator 105, a detector assembly 108, and the like are attached to the gantry 102.

The X-ray tube 104 generates X-rays when a prescribed voltage is applied to a cathode-anode tube. The X-ray tube 104 is configured to be rotatable on a path centered on a rotation axis 206 within the XY plane. Herein, the Z direction represents the body-axis direction, the Y direction represents the vertical direction (the height direction of the table 116), and the X direction represents the direction perpendicular to the Z and Y directions. In the present embodiment, the X-ray tube 104 supports a kV switching scheme in which the tube voltage applied to the X-ray tube 104 can be switched between a first tube voltage and a second tube voltage. Note that in the present embodiment, the CT system 100 includes one X-ray tube 104, but may include two X-ray tubes 104.

The filter part 103 includes, for example, a flat plate filter and/or a bow-tie filter. The pre-collimator 105 is a member that narrows the X-ray irradiation range such that X-rays are not irradiated in unwanted regions.

The detector assembly 108 includes a plurality of detector elements 202. The plurality of detector elements 202 detect an X-ray beam 106 that is irradiated from the X-ray tube 104 and passes through the subject 112, such as a patient or the like. Therefore, the detector assembly 108 can acquire projection data for each view.

The projection data detected by the detector assembly 108 is collected by a DAS 214. The DAS 214 executes prescribed processing, including sampling, digital conversion, and the like, on the collected projection data. The processed projection data is transmitted to a computer 216. The computer 216 stores the data from the DAS 214 in a storing device 218. The storing device 218 includes one or more recording medium that stores a program, an instruction to be executed by a processor, and the like. The recording medium may be, for example, one or more non-transitory computer-readable recording medium. The storing device 218 may include, for example, hard disk drives, floppy disk drives, compact disc read/write (CD-R/W) drives, digital versatile disk (DVD) drives, flash drives, and/or solid state recording drives.

The computer 216 includes one or a plurality of processors 217. The computer 216 uses the one or plurality of processors 217 to output commands and parameters to the DAS 214, X-ray controller 210, and/or gantry motor controller 212, to control system operations such as data acquisition and/or processing. Furthermore, the computer 216 uses one or a plurality of processors to execute various processes such as signal processing, data processing, image processing, and the like in each step of the flow described later. Note that in FIG. 1, one or a plurality of the processors 217 are included in the computer 216, but one or a plurality of the processors 217 may be provided so as to be distributed between the computer 216 and another component (e.g., X-ray controller 210, gantry motor controller 212, table motor controller 118, or the like).

An operator console 220 is linked to the computer 216. An operator can enter prescribed operator inputs related to the operation of the CT system 100 into the computer 216 by operating the operator console 220. The computer 216 receives an operator input, including a command and/or scan parameter, via the operator console 220 and controls system operation based on the operator input. The operator console 220 can include a keyboard (not depicted) or touch screen for the operator to specify a command and/or scan parameter.

The X-ray controller 210 controls the X-ray tube 104 based on an instruction from the computer 216. Furthermore, the gantry motor controller 212 controls a gantry motor to rotate a component, such as the X-ray tube 104, detector assembly 108, or the like, based on an instruction from the computer 216.

FIG. 1 depicts only one operator console 220, but two or more operator consoles may be linked to the computer 216. Furthermore, the CT system 100 may also allow a plurality of remotely located displays, printers, workstations, and/or similar devices to be linked via, for example, a wired and/or wireless network.

In one embodiment, for example, the CT system 100 may include a Picture Archiving and Communication System (PACS) 224, or may be linked to the PACS 224. In a typical implementation, a PACS 224 may be linked to a remote system such as a radiology department information system, hospital information system, and/or internal or external network (not depicted) or the like.

The computer 216 provides an instruction to a table motor controller 118 to control the table 116. The table motor controller 118 can control the table motor so as to move the table 116 based on the instructions received. For example, the table motor controller 118 can move the table 116 such that the subject 112 is positioned appropriately for imaging.

As mentioned above, the DAS 214 samples and digitally converts the projection data acquired by the detector elements 202. The image reconstructor 230 then reconstructs the image using the sampled and digitally converted data. The image reconstructor 230 includes one or a plurality of processors, which can execute image reconstruction processing. In FIG. 1, the image reconstructor 230 is depicted as a separate component from the computer 216, but the image reconstructor 230 may form a part of the computer 216. Furthermore, the computer 216 may also perform one or a plurality of functions of the image reconstructor 230. Furthermore, the image reconstructor 230 may be positioned away from the CT system 100 and operatively connected to the CT system 100 using a wired or wireless network.

The image reconstructor 230 can store the reconstructed image in the storing device 218. The image reconstructor 230 may also transmit the reconstructed image to the computer 216. The computer 216 can transmit the reconstructed image and/or patient information to a display device 232 communicatively linked to the computer 216 and/or image reconstructor 230.

The various methods and processes described in the present specification can be recorded as executable instructions on a non-transitory recording medium within the CT system 100. The executable instructions may be stored on a single recording medium or distributed across a plurality of recording media. One or more processors provided in the CT system 100 execute the various methods, steps, and processes described in the present specifications in accordance with the instructions recorded on a recording medium.

The CT system 100 is configured as described above. In the present embodiment, the CT system is configured to be able to image a subject using kV switching technology, which switches the tube voltage of the X-ray tube between a high tube voltage (hereinafter referred to as “high kV”) and a low tube voltage (hereinafter referred to as “low kV”).

In the kV switching technology, data acquired by the detector must be separated into data corresponding to the high kV and data corresponding to the low kV. In general, if the voltage difference between the high kV and low kV is small, the accuracy of identifying substances (accuracy of identifying data) tends to deteriorate. Therefore, in the kV switching technology, the tube voltage must be switched such that the difference between the high kV and low kV is as large as possible.

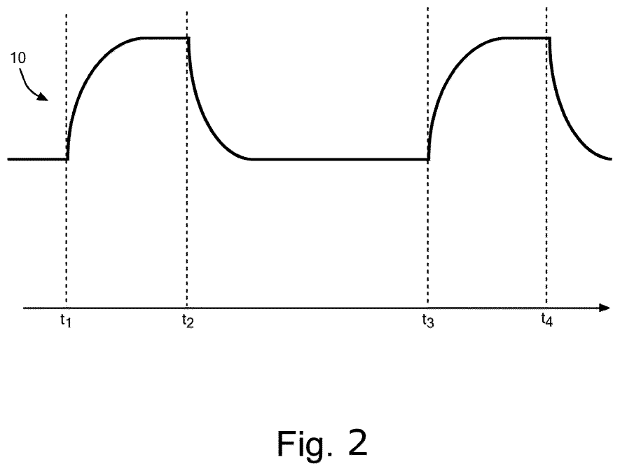

FIG. 2 is an explanatory diagram when switching the tube voltage. FIG. 2 depicts a tube voltage waveform 10. Immediately before time t1, a low kV is applied to the X-ray tube. At the time t1, the tube voltage applied to the X-ray tube 104 switches from low kV to high kV. Ideally, the tube voltage switches in a rectangular pattern. However, in reality, due to a transient phenomenon in the tube voltage, it is not possible to instantly reach a high kV from a low kV at the time t1, and a certain amount of time is required to reach a prescribed voltage value.

Furthermore, at time t2, which is a prescribed time after the time t1, the tube voltage of the X-ray tube is switched from high kV to low kV. However, due to the transient phenomenon of the tube voltage, it is not possible to instantaneously reach a low kV from a high kV at the time t2, and a certain amount of time is required to reach a prescribed voltage value.

Next, at time t3, which is a prescribed time after the time t2, the tube voltage of the X-ray tube is switched from low kV to high kV. However, due to the transient phenomenon of the tube voltage, it is not possible to instantaneously reach a high kV from a low kV at the time t3, and a certain amount of time is required to reach a prescribed voltage value.

Hereinafter, switching between high kV and low kV is performed in the same manner. Therefore, the tube voltage waveform includes a high kV period and a low kV period.

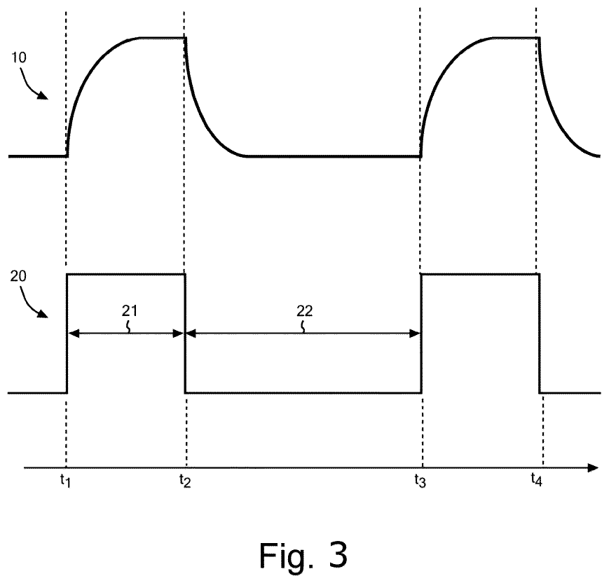

FIG. 3 is an explanatory diagram of the high kV period and the low kV period of the tube voltage waveform. The upper part of FIG. 3 depicts the tube voltage waveform 10 depicted in FIG. 2, and the lower part depicts a rectangular waveform 20. The rectangular waveform 20 represents a period 21 corresponding to a high kV and a period 22 corresponding to a low kV. The period 21 represents a period from the time t1 when the tube voltage starts to switch from a low kV to a high kV to the time t2 when the tube voltage starts to switch from the high kV to the low kV.

On the other hand, a period 22 represents a period from the time t2 when the tube voltage starts to switch from high kV to low kV to the time t3 when the tube voltage starts to switch from low kV to high kV.

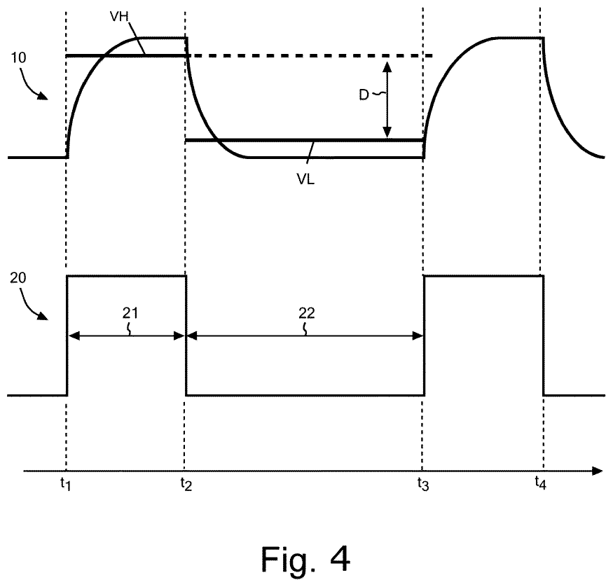

In the example above, the period 21 represents a high kV period and period 22 represents a low kV period. However, the tube voltage does not vary in a rectangular fashion as depicted in FIG. 3, but varies in accordance with constant rise and fall times. Therefore, the period 21 includes not only a voltage that is sufficiently close to high kV, but also a voltage immediately after switching from low kV to high kV. For this reason, when considering the tube voltage during the period 21, the effective voltage value must be considered, rather than the ideal value of the tube voltage. Similarly, the period 22 includes not only a voltage that is sufficiently close to low kV, but also a voltage immediately after switching from high kV to low kV. Therefore, when considering the tube voltage during the period 22, the effective voltage value must also be considered, rather than the ideal value of the tube voltage. FIG. 4 depicts an example of an effective voltage value VH corresponding to the tube voltage in the period 21 and an effective voltage value VL corresponding to the tube voltage in the period 22.

In the kV switching technology, data acquired by the detector must be separated into data corresponding to the high kV and data corresponding to the low kV. In general, if an effective voltage difference D between the effective voltage values VH and VL is small, the accuracy of identifying substances (accuracy of identifying data) tends to be low, and thus the effective voltage difference D needs to be as large as possible. However, in the example of FIG. 4, the period 21 includes not only a voltage that is sufficiently close to high kV, but also the voltage immediately after switching from low kV to high kV, and thus the effective voltage value VH is influenced by the voltage immediately after switching from low kV to high kV and cannot be made to a sufficiently large value. Similarly, the period 22 includes not only a voltage that is sufficiently close to low kV, but also the voltage immediately after switching from high kV to low kV, and thus the effective voltage value VL is influenced by the voltage immediately after switching from high kV to low kV and cannot be made to a sufficiently small value. For this reason, the effective voltage difference D cannot be made sufficiently large. Therefore, in order to deal with this problem, a certain delay time is provided between when the tube voltage is changed and when projection data collection starts. Next, the delay time will be described.

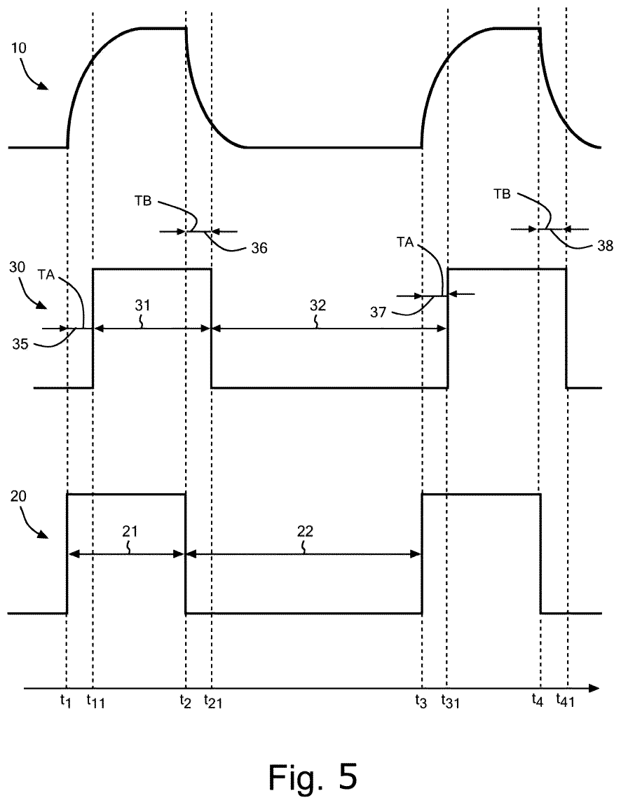

FIG. 5 is an explanatory diagram of the delay time. FIG. 5 depicts the tube voltage waveform 10, and a rectangular waveform 30 with a delay time provided is depicted below the tube voltage waveform 10. Note that for reference, the rectangular waveform 20 with no delay time is also depicted below the rectangular waveform 30.

The rectangular waveform 30 is provided with a first delay time TA and a second delay time TB with respect to the rectangular waveform 20. Due to space limitations, FIG. 5 only depicts two first delay times TA (i.e., a first delay time TA in period 35 and a first delay time TA in period 37) and two second delay times TB (i.e., a second delay time TB in period 36 and a second delay time TB in period 38).

The delay time TA of the period 35 is from the time t1 when starting to switch from low kV to high kV to time t11 when projection data collection starts. Furthermore, the delay time TB of the period 36 is from the time t2 when starting to switch from high kV to low kV to time t21 when projection data collection starts. Therefore, the detector assembly 108 detects projection data corresponding to high kV during the period 31 (i.e., period offset from the period 21 by delay times TA and TB).

Furthermore, the delay time TA of the period 37 is from the time t3 when starting to switch from low kV to high kV to time t31 when projection data collection starts. Therefore, the detector assembly 108 detects projection data corresponding to low kV during the period 32 (i.e., period offset from the period 22 by delay times TB and TA).

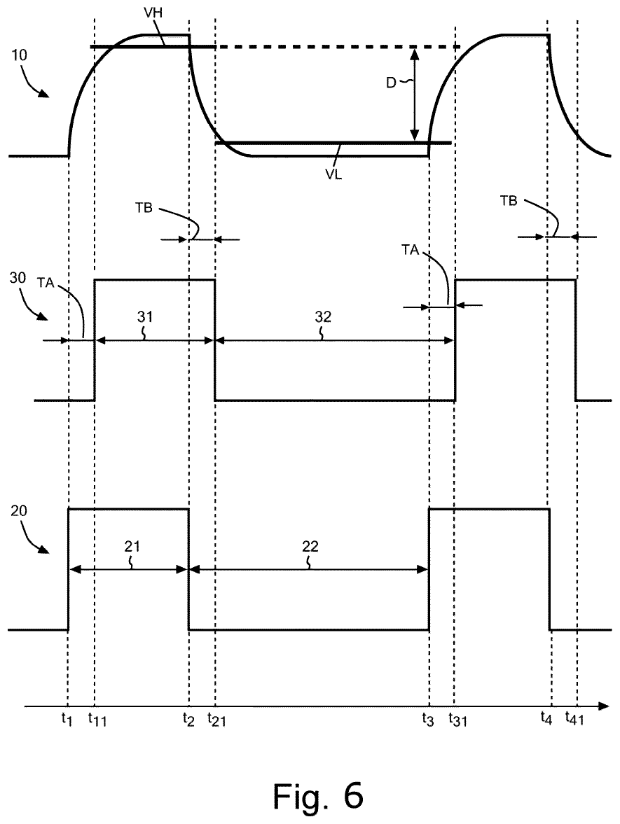

FIG. 6 is a diagram depicting an effective voltage difference obtained for the rectangular waveform 30. The period 31 may have a wider range than the period 21, including high kV (and voltages close to high kV). Therefore, the effective voltage value VH can be increased. Furthermore, the period 32 may have a wider range than the period 22, including low kV (and voltages close to low kV). Therefore, the effective voltage value VL can be reduced. For this reason, by providing the first delay time TA and the second delay time TB, the effective voltage difference D can be increased.

The specific values of the first delay time TA and the second delay time TB are generally determined as fixed values during the development stage of the CT system through experiments or the like, taking into consideration individual differences and deterioration over time of the CT system itself. Furthermore, delay times determined at the development stage are used as default values for the first delay time TA and the second delay time TB. However, the optimal delay times vary depending on individual differences of the CT system itself. Furthermore, the optimal delay time changes due to aging of parts used in the CT system and the like. Therefore, if the delay time set as the default value is continued to be used, there is a problem where acquisition of a high-quality image becomes difficult.

Therefore, the CT system of the present embodiment is configured such that the optimal delay time can be obtained even after the CT system is installed in a facility such as a hospital or the like. A method for obtaining an optimal delay time will be described below.

Note that in the present embodiment, as described later, the effective voltage value VH corresponding to a high kV and the effective voltage value VL corresponding to a low kV are obtained, and optimal values of the first delay time TA and the second delay time TB are obtained based on the effective voltage values VH and VL. Therefore, in order to find the optimal values of the first delay time TA and the second delay time TB, the effective voltage values VH and VL must be obtained. Therefore, first, a method for obtaining the effective voltage values VH and VL in the present embodiment will be described below. Furthermore, after this description, a method for obtaining the optimal values of the first delay time TA and the second delay time TB will be described.

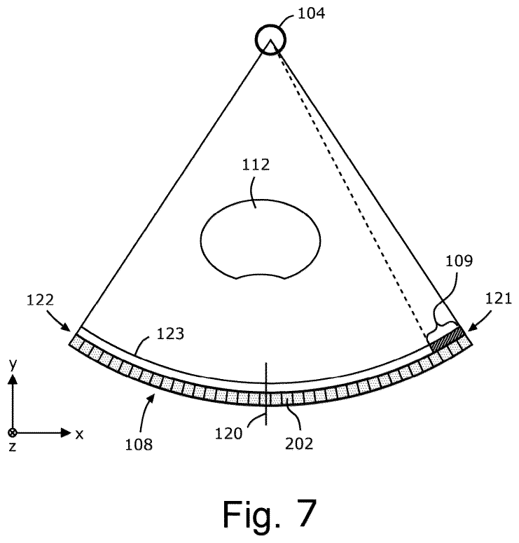

FIGS. 7 to 12 are explanatory diagrams of a method for calculating the effective voltage value in the present embodiment. FIG. 7 is an explanatory diagram of the detector assembly 108.

The detector assembly 108 includes a plurality of the detector elements 202. The detector assembly 108 is provided with a reference region 109. Energy information of X-rays detected in the reference region 109 is used to calculate an index value M, which will be described later. The index value M will be described in detail later. The reference region 109 is provided at a position as far away as possible from a center position 120 in the X direction of the detector assembly 108 such that, during scanning of the subject 112, X-rays irradiated from the X-ray tube 104 that do not pass through the subject 112 can be detected. In the present embodiment, the reference region 109 is provided at one end part 121 of the detector assembly 108. Note that the reference region 109 may be provided not only at the one end part 121 of the detector assembly 108 but also at an opposite end part 122 of the detector assembly 108. In the present embodiment, the description will be continued with the reference region 109 being provided only at the one end part 121 of the detector assembly 108.

Furthermore, the detector assembly 108 includes a detector collimator 123 to reduce image quality deterioration caused by scattered radiation. The detector collimator 123 is disposed on the X-ray tube 104 side with respect to the detector elements 202.

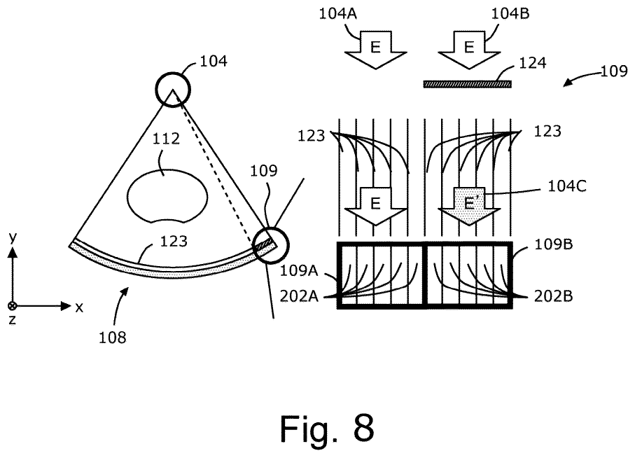

FIG. 8 is an enlarged view of the reference region 109 of the detector assembly 108. The detector collimator 123 is provided on an extension of a boundary between adjacent detector elements 202.

Furthermore, FIG. 8 depicts X-rays 104A and 104B. The X-rays 104A represent X-rays advancing toward a detector element 202A of the plurality of detector elements 202 disposed in the reference region 109 of the detector assembly 108. Furthermore, the X-rays 104B represent X-rays advancing toward a detector element 202B of the plurality of detector elements 202 disposed in the reference region 109 of the detector assembly 108.

The detector assembly 108 includes a filter 124. The filter 124 may be manufactured using, for example, copper, molybdenum, tungsten, or the like. The filter 124 is not disposed in the path of the X-rays 104A, but is disposed in the path of the X-rays 104B. The X-rays 104A are detected by the detector element 202A without passing through filter 124. On the other hand, the X-rays 104B pass through the filter 124, and X-rays 104C, the energy of which has been absorbed by the filter 124, are detected by the detector element 202B. Therefore, the reference region 109 of the detector assembly 108 has a first sub-region 109B in which X-rays that have passed through the filter 124 are detected and a second sub-region 109A in which X-rays that have not passed through the filter 124 are detected.

In the present embodiment, as described above, the CT system 100 includes the detector assembly 108 having the filter 124 in the reference region 109, and the effective voltage value of the X-ray tube 104 can be obtained based on the X-rays detected in the reference region 109 of the detector assembly 108. However, in order to obtain the effective voltage value, a baseline that is used to obtain the effective voltage value must be prepared in advance. The baseline can generally be created when installation work is performed to install the CT system 100 in a facility such as a hospital or the like. A method for creating a baseline will be described below.

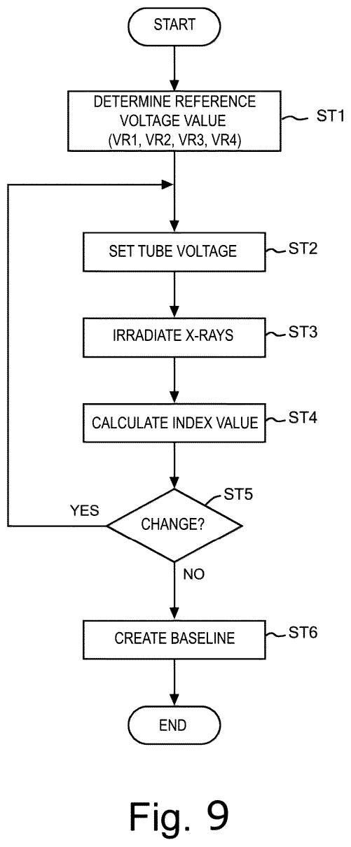

FIG. 9 is a flowchart of the method for creating a baseline. In step ST1, a reference voltage (hereinafter referred to as “reference tube voltage”) for a tube voltage used to create a baseline is determined. In the present embodiment, four reference tube voltages VR1, VR2,VR3, and VR4 are determined as reference tube voltages. The reference tube voltages VR1 to VR4 can be determined based on tube voltages actually used in kV switching. In the present embodiment, the reference tube voltages are VR1=80 kV, VR2=100 kV, VR3=120 kV, and VR4=140 kV, but other reference tube voltages may be adopted.

Note that in the present embodiment, the four reference tube voltages VR1 to VR4 are determined as the reference tube voltages used to create the baseline. However, five or more reference tube voltages may be determined as the reference tube voltages used to create the baseline, or three or less reference tube voltages may be determined as the reference tube voltages used to create the baseline. Note that if the number of reference tube voltages is too small, the reliability of the accuracy of the effective tube voltage may decrease. On the other hand, if the number of reference tube voltages is too large, a great deal of time is required for data collection. Thus, the number of reference tube voltages is preferably determined in consideration of a balance thereof. As an example, the four reference tube voltages VR1 to VR4 employed in the present embodiment are preferably determined as the reference tube voltages used to create the baseline. After determining the four reference tube voltages VR1 to VR4, the process proceeds to step ST2.

In step ST2, the tube voltage of the X-ray tube 104 is set to a reference tube voltage used to create the baseline. Herein, of the four reference tube voltages VR1 to VR4 determined in step ST1, the reference tube voltage VR1 (=80 kV) is first selected, and the tube voltage of the X-ray tube 104 is set to the reference tube voltage VR1 (=80 kV). Once the reference tube voltage is set, the process proceeds to step ST3.

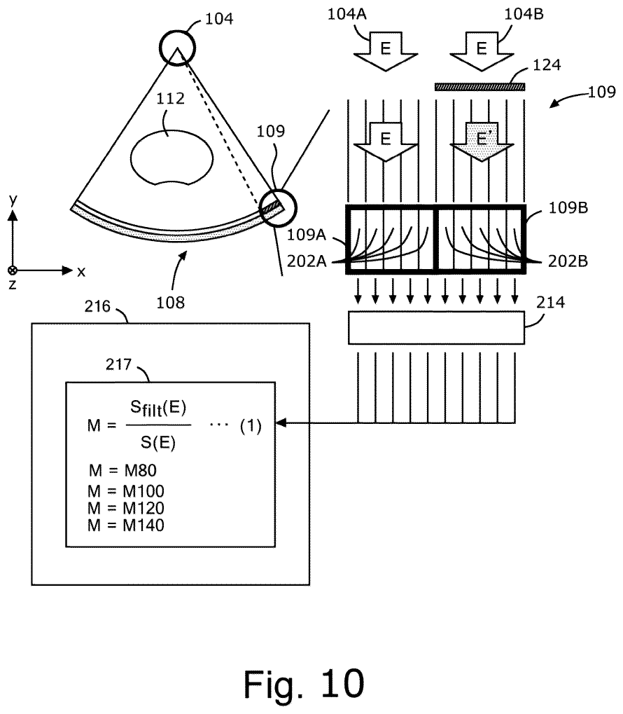

In step ST3, the reference tube voltage VR1 (=80 kV) is applied to the X-ray tube 104, and X-rays are irradiated from the X-ray tube 104. The X-rays irradiated from the X-ray tube 104 are detected by the detector assembly 108. The X-rays detected by the detector assembly 108 are output as an analog signal to the DAS 214 as depicted in FIG. 10. The DAS 214 samples the analog data received from detector assembly 108 and converts the analog data to a digital signal for subsequent processing. The digital signal is output to the computer 216.

In step ST4, the computer processor 217 calculates, based on the digital signal, an index value M representing the difference between energy information of X-rays detected in the first sub-region 109B of the reference region 109 (i.e., X-rays that have passed through the filter 124) and energy information of X-rays detected in the second sub-region 109A of the reference region 109 (i.e., X-rays that have not passed through the filter 124). A method for calculating the index value M will be described with reference to FIG. 10.

In the present embodiment, the index value M can be calculated by the following equation:

$$M = Sfilt ( E ) / S ( E ) \qquad ( 1 )$$

Herein, M: index value
Sfilt(E): Signal value including energy information of X-rays detected by the detector element 202B of the first sub-region 109B (i.e., X-rays that have passed through the filter 124)
S(E): Signal value including energy information of X-rays detected by the detector element 202A of the second sub-region 109A (i.e., X-rays that have not passed through the filter 124)

Therefore, the index value M can be expressed as a ratio between the signal value Sfilt (E) including energy information of X-rays that have passed through the filter 124 and been detected in the first sub-region 109B and the signal value S(E) including energy information of X-rays that have not passed through the filter 124 and have been detected in the second sub-region 109A.

Note that the signal value Sfilt(E) can be obtained based on a signal obtained from one or more detector element 202B of the five detector elements 202B. For example, the signal value Sfilt(E) may be obtained based on a representative value (maximum value, integral value, or the like) of a signal obtained from one detector element 202B of the five detector elements 202B, or the like, or obtained based on the average value and/or total value of representative values (maximum values, integral values, or the like) of signals obtained from two or more detector elements 202B of the five detector elements 202B, or the like.

Similarly, the signal value S(E) can be obtained based on a signal obtained from one or more detector element 202A of the five detector elements 202A. For example, the signal value S(E) may be obtained based on a representative value (maximum value, integral value, or the like) of a signal obtained from one detector element 202A of the five detector elements 202A, or the like, or obtained based on the average value and/or total value of representative values (maximum values, integral values, or the like) of signals obtained from two or more detector elements 202A of the five detector elements 202A, or the like.

Therefore, by setting the tube voltage of the X-ray tube 104 to 80 kV and obtaining the signal values Sfilt(E) and S(E) based on the energy of X-rays detected in the reference region 109 of the detector assembly 108, the index value M (=M80) corresponding to the reference tube voltage VR1 (=80 kV) can be calculated from equation (1).

As described above, when calculating the index value M80, the tube voltage of the X-ray tube 104 is constant at 80 kV. Therefore, there is no need to switch the tube voltage between high and low kVs while the gantry is rotating, and thus the value of the tube voltage remains stable while the gantry is rotating. For this reason, the index value M80 can be calculated without being affected by the rotation speed of the gantry. Furthermore, the index value M80 is a value calculated based on the X-ray energy information. The energy of the X-rays basically depends on the tube voltage of the X-ray tube 104 and does not depend so much on the tube current, and therefore, the index value M80 can be calculated without being affected by the tube current.

Thus, the index value M80 corresponding to the reference tube voltage VR1 (=80 kV) can be calculated without being substantially affected by the rotation speed of the gantry or the tube current.

After calculating the index value M80, the index value M80 is stored in the storing device, and the process proceeds to step ST5.

In step ST5, whether or not to change the reference tube voltage is determined. Herein, of the four reference tube voltages VR1 to VR4, only the index value M80 of the reference tube voltage VR1 is calculated. Therefore, the process returns to step ST2 to calculate the index values M of the remaining reference tube voltages.

In step ST2, the reference tube voltage of the X-ray tube 104 is set to another voltage value. Herein, of the four reference tube voltages VR1 to VR4 determined in step ST1, the reference tube voltage VR2 (=100 kV) is selected, and the tube voltage of the X-ray tube 104 is set to the reference tube voltage VR2 (=100 kV).

In step ST3, the reference tube voltage VR2 (=100 kV) is applied to the X-ray tube 104, and X-rays are irradiated from the X-ray tube 104. The X-rays irradiated from the X-ray tube 104 are detected by the detector assembly 108. The X-rays detected by the detector assembly 108 are output as an analog signal to the DAS 214 as depicted in FIG. 5. The DAS 214 samples the analog data received from detector assembly 108 and converts the analog data to a digital signal for subsequent processing. The digital signal is output to the computer 216.

In step ST4, the processor 217 calculates an index value M100 corresponding to the reference tube voltage VR2 using equation (1), and stores the index value M100 in the storing device. Furthermore, the process proceeds to step ST5, and whether or not to change the reference tube voltage is determined. Herein, of the four reference tube voltages VR1 to VR4, the index values M80 and M100 of the two reference tube voltages VR1 and VR2 have been calculated, but the index values for the remaining two reference tube voltages VR3 and VR4 have not yet been calculated. Therefore, the process returns to step ST2 to calculate the index values of the remaining reference tube voltages.

Similarly, the loop of steps ST2 to ST5 is repeatedly executed until an index value M120 corresponding to the reference tube voltage VR3 (=120 kV) and an index value M140 corresponding to the reference tube voltage VR4 are calculated. Furthermore, once the index value M140 corresponding to the reference tube voltage VR4 has been calculated, the process proceeds to step ST5.

In step ST5, whether or not to change the reference tube voltage is determined. Herein, the index values M80 to M140 corresponding to all of the four reference tube voltages VR1 to VR4 are calculated. Therefore, the process proceeds to step ST6.

In step ST6, a baseline used to determine the effective voltage value is created based on the four calculated index values M80 to M140.

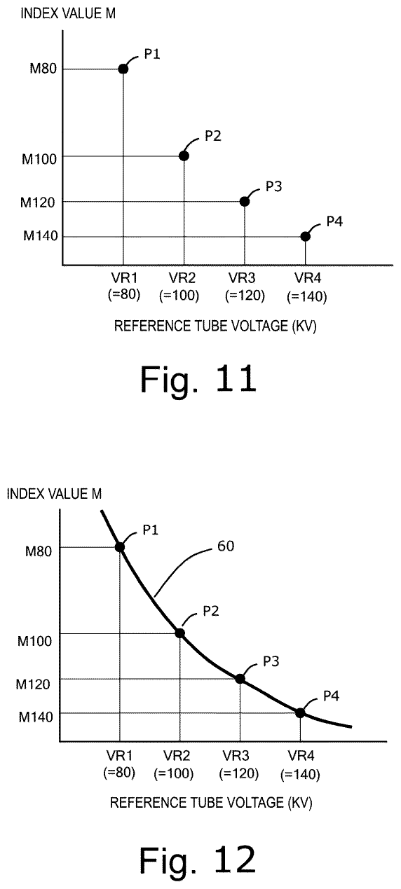

FIG. 11 is an explanatory diagram of a method for creating a baseline. The computer processor 217 associates the reference tube voltages VR1 to VR4 with the index values M80 to M140 on a two-dimensional coordinate system.

Point P1 is a point that associates the reference tube voltage VR1 with the index value M80 calculated for the reference tube voltage VR1. Point P2 is a point that associates the reference tube voltage VR2 with the index value M100 calculated for the reference tube voltage VR2. Point P3 is a point that associates the reference tube voltage VR3 with the index value M120 calculated for the reference tube voltage VR3. Point P4 is a point that associates the reference tube voltage VR4 with the index value M140 calculated for the reference tube voltage VR4.

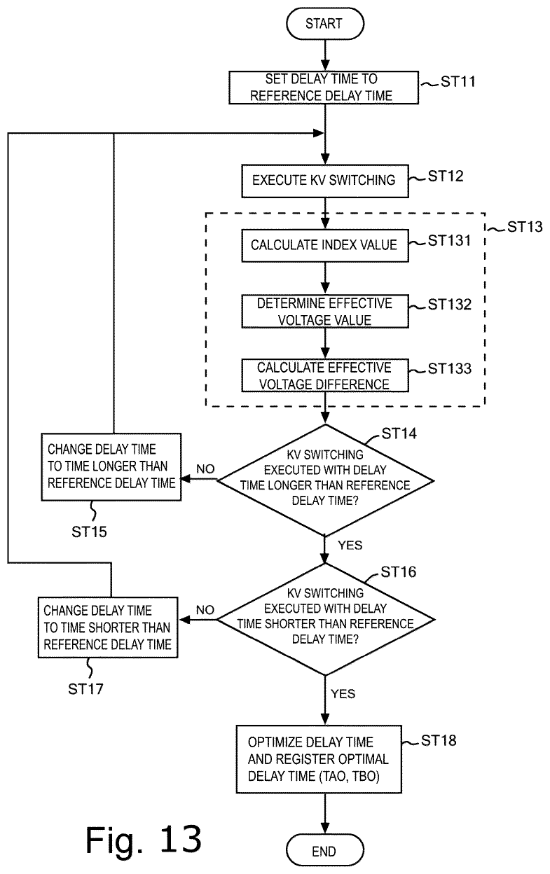

Furthermore, the processor 217 creates a baseline representing the relationship between the tube voltage and the index value M based on the points P1 to P4. The baseline can be obtained, for example, as a curve that optimally fits points P1 to P4. FIG. 12 depicts a baseline 60 obtained thereby. Data representing the baseline 60 may be stored in a storing device of the CT system 100 or in an external storing device capable of communicating with the CT system 100.

A method for obtaining an optimal delay time using a baseline will be described below.

Note that in general, two cases are considered as examples in which it is desirable to execute work for obtaining the optimal delay time. The first example is a case for executing installation work to install the CT system in a facility such as a hospital or the like, executing maintenance, or replacing a part. The second example is a case in which a user of the CT system periodically executes simple calibration of the CT system after the CT system installed in the facility actually starts to operate. Therefore, in the following description, a method for obtaining the optimal delay time will be described for each of the first and second examples.

Note that as the first example, the installation work of installing the CT system in a facility such as a hospital or the like will be used and described.

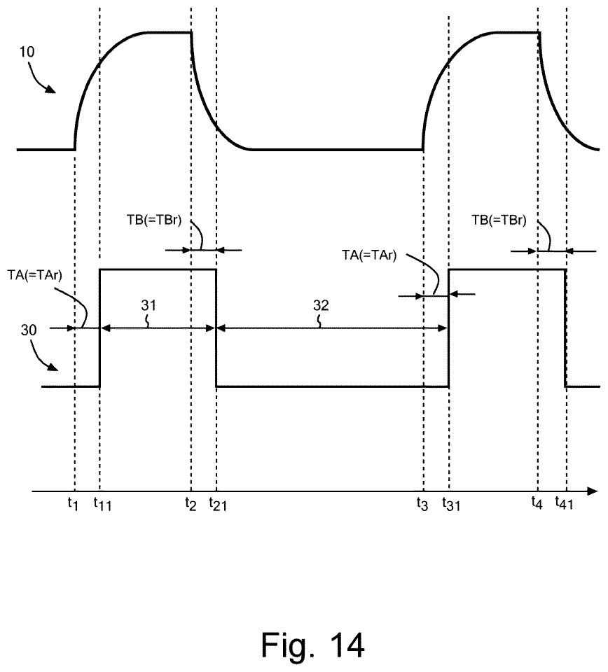

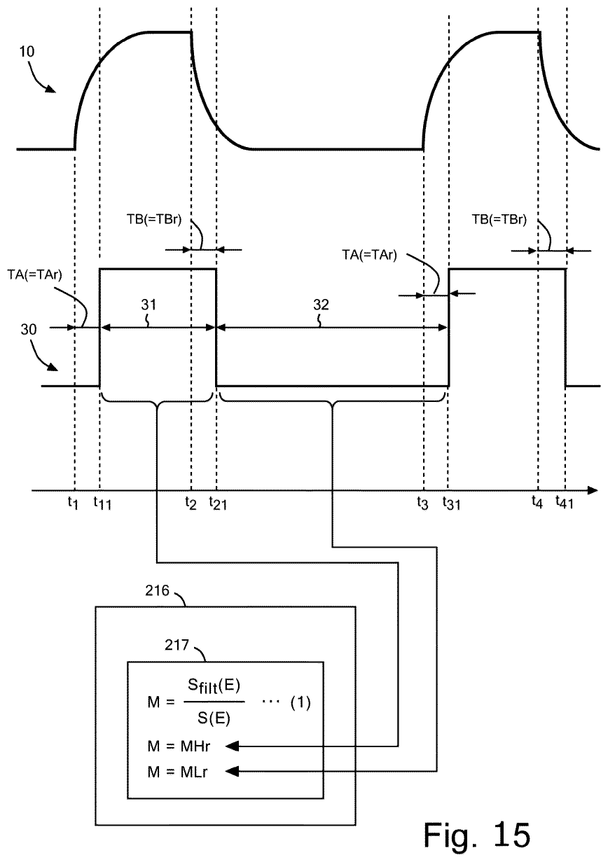

FIG. 13 is a flowchart for a method for obtaining the optimal delay time during installation work of the CT system. In step ST11, the processor 217 sets the first delay time TA to a first reference delay time TAr, and sets the second delay time TB to a second reference delay time TBr, as depicted in FIG. 14. The first reference delay time TAr is a delay time that represents a reference value of the first delay time TA, and the second reference delay time TBr is a delay time that represents a reference value of the second delay time TB. The reference delay times TAr and TBr are values that are determined in advance at a stage prior to the installation work of the CT system, and are values that are stored as default values in a storing device or an external storing device. The reference delay time (TAr, TBr) may be a value determined through experiments or the like in the development stage of the CT system. Note that the first reference delay time TAr and the second reference delay time TBr may be the same value or different values. The processor 217 reads the reference delay times TAr and TBr from the storing device, and sets the first delay time TA to the first reference delay time TAr and sets the second delay time TB to the second reference delay time TBr, as depicted in FIG. 14. After setting the reference delay time, the process proceeds to step ST12.

In step ST12, in a state in which the delay time (TA, TB) is set to the reference delay time (TAr, TBr), kV switching is executed, in which the tube voltage is alternately switched between high kV and low kV, and X-rays are detected by the detector assembly 108. As an example, the high kV may be 140 kV and the low kV may be 80 kV. The DAS 214 executes prescribed processing, including sampling processing or the like, on data including the energy information of the detected X-rays to convert the data into digital data, which is then output to a computer.

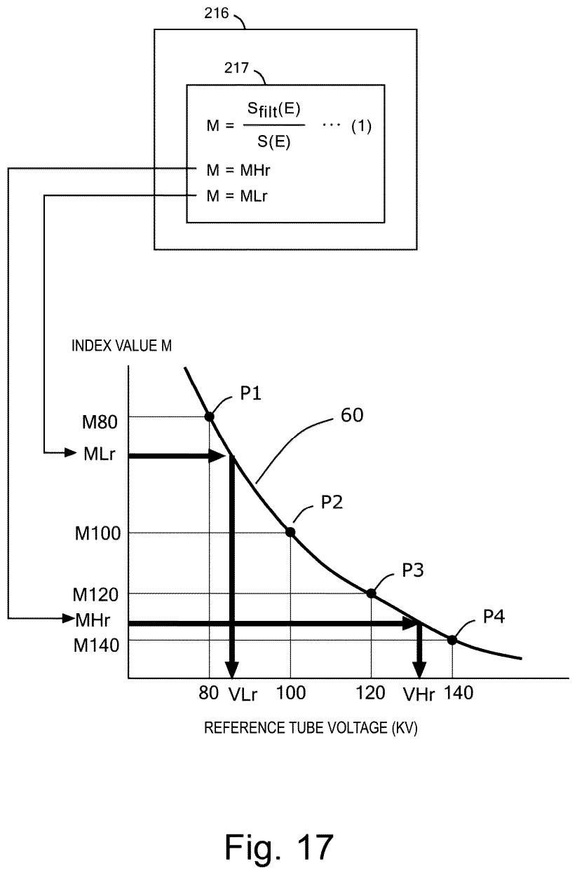

In step ST13, the computer processor 217 calculates a first effective voltage difference D1 (see FIG. 16) based on the data received from the DAS 214. Note that step ST13 includes steps ST131 to ST133, and thus each step will be described in order.

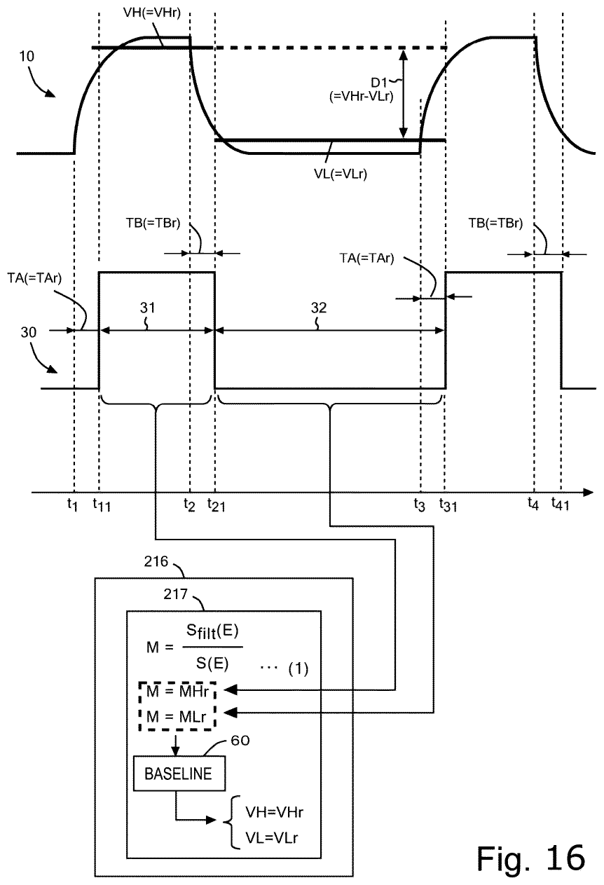

In step ST131, the computer processor 217 determines signal values Sfilt(E) and S(E) included in equation (1) based on the data received from the DAS 214, and calculates the index value M (=MHr) corresponding to high kV and the index value M (=MLr) corresponding to low kV (see FIG. 15).

FIG. 15 is an explanatory diagram of a method for calculating the index value M (=MHr) corresponding to high kV and the index value M (=MLr) corresponding to low kV. The processor 217 determines the signal values Sfilt(E) and S(E) in equation (1) based on the data obtained from the reference region 109 (see FIG. 10) during the period 31 in which high kV data is collected, and substitutes the determined signal values Sfilt(E) and S(E) into equation (1) to calculate the index value M (=MHr) corresponding to high kV. Note that in a kV switching scan, when N-number of periods 31 appear during which high kV data is collected, the signal values Sfilt(E) and S(E) may be determined based on all data obtained in the N-number of periods 31, or may be determined based on data obtained in M−(<N) number of periods 31 out of the N-number of periods 31. The determined signal values Sfilt(E) and S(E) are substituted into equation (1) to calculate the index value M (=MHr) corresponding to high kV.

Furthermore, the processor 217 determines the signal values Sfilt(E) and S(E) in equation (1) based on the data obtained from the reference region 109 during the period 32 in which low kV data is collected, and substitutes the determined signal values Sfilt(E) and S(E) into equation (1) to calculate the index value M (=MLr) corresponding to low kV. Note that in a kV switching scan, when N-number of periods 32 appear during which low kV data is collected, the signal values Sfilt(E) and S(E) may be determined based on all data obtained in the N-number of periods 32, or may be determined based on data obtained in M−(<N) number of periods 32 out of the N-number of periods 32.

After calculating the index value M (=MHr) corresponding to high kV and the index value M (=MLr) corresponding to low kV, the process proceeds to step ST132.

In step ST132, the processor 217 determines a first effective voltage value corresponding to high kV based on the index value M (=MHr), and determines a second effective voltage value corresponding to low kV based on the index value M (=MLr) (see FIG. 16).

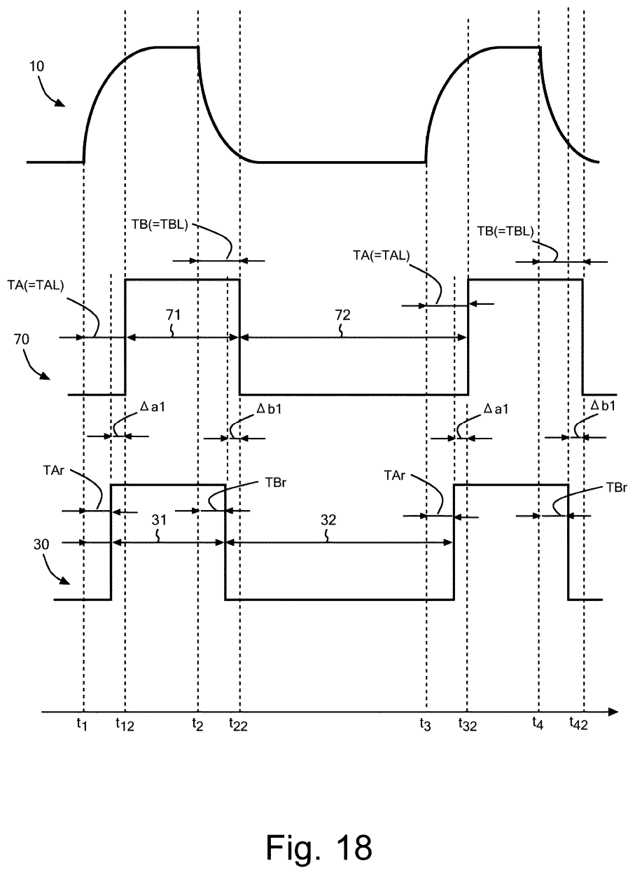

FIG. 16 is an explanatory diagram of a method for determining an effective voltage value. The processor 217 reads the baseline 60 stored in the storing device. Furthermore, based on the baseline 60, the processor 217 determines the first effective voltage value VH (=VHr) corresponding to the high-kV index value M (=MHr) and the second effective voltage value VL (=VLr) corresponding to the low-kV index value M (=MLr) (see FIG. 17).

FIG. 17 is a specific explanatory diagram of a method for determining effective voltage values VHr and VLr using the baseline 60. The baseline 60 represents the relationship between the index value M and the tube voltage. Therefore, the processor 217 can identify the first effective voltage value VH (=VHr) corresponding to the high-kV index value M (=MHr) based on the baseline 60. The effective voltage value VHr is determined as the effective voltage value corresponding to high kV.

Furthermore, the processor 217 can identify the second effective voltage value VL (=VLr) corresponding to the low-kV index value M (=MLr) based on the baseline 60. The effective voltage value VLr is determined as the effective voltage value corresponding to low kV.

Returning to FIG. 16, the tube voltage waveform 10 indicates the effective voltage value VHr and the effective voltage value VLr determined using the baseline 60. After determining the effective voltage value VHr and the effective voltage value VLr, the process proceeds to step ST133.

In step ST133, the processor 217 calculates the first effective voltage difference D1 representing the difference between the first effective voltage value VH (=VHr) corresponding to high kV and the second effective voltage value VL (=VLr) corresponding to low kV. The first effective voltage difference D1 is, as depicted in FIG. 16, D1=VHr−VLr. The effective voltage difference D1 is stored in the storing device in association with the effective voltage value (VHr, VLr) and the reference delay time (TAr, TBr). After calculating the first effective voltage difference D1, the process proceeds to step ST14.

In step ST14, the processor 217 determines whether or not kV switching has been executed with a delay time longer than the reference delay time. Herein, kV switching was executed with the delay time set to the reference delay time, but kV switching was not executed with a delay time longer than the reference delay time. Therefore, the process proceeds to step ST15.

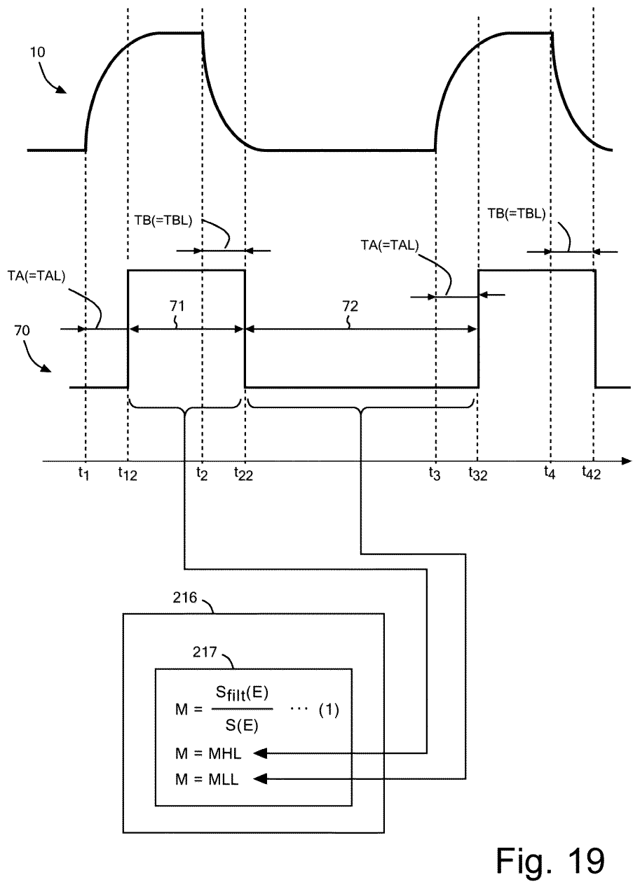

In step ST15, the processor 217 sets the delay time to a time longer than the reference delay time (see FIG. 18). FIG. 18 is a diagram depicting an example in which the delay time is set to a time longer than the reference delay time. FIG. 18 depicts the tube voltage waveform 10, and a rectangular waveform 70, in which the delay time is longer than the reference delay time, is depicted below the tube voltage waveform 10. Note that for reference, the rectangular waveform 30, in which the delay time is set to the reference delay time, is depicted below the rectangular waveform 70.

In the rectangular waveform 70, the delay time TA is set to a delay time TAL that is longer than the first reference delay time TAr by Δa1. Furthermore, the delay time TB is set to a delay time TBL that is longer than the second reference delay time TBr by Δb1. Note that the long delay time TAL and the long delay time TBL may be the same value or different values. Once the delay time is set to a time (TAL, TBL) longer than the reference delay time (TAr, TBr), the process returns to step ST12.

In step ST12, in a state in which the delay time (TA, TB) is set to a time (TAL, TBL) longer than the reference delay time (TAr, TBr), kV switching in which the tube voltage is alternately switched between high kV and low kV is executed, and X-rays are detected by the detector assembly 108. The DAS 214 executes prescribed processing, including sampling processing or the like, on data including the energy information of the detected X-rays to convert the data into digital data, which is then output a computer.

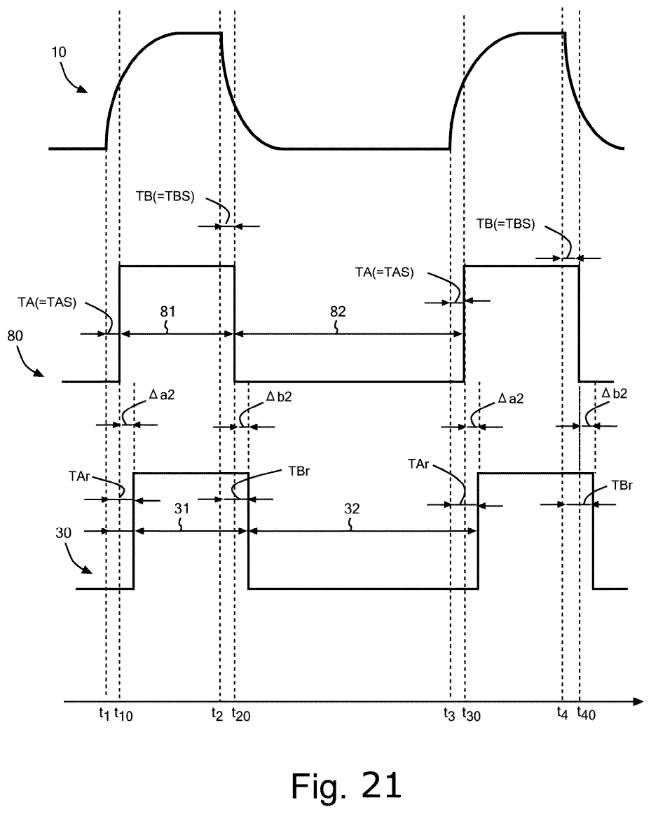

In step ST13, the computer processor 217 calculates a second effective voltage difference D2 (see FIG. 20) based on the data received from the DAS 214. Specifically, the second effective voltage difference D2 is calculated as follows.

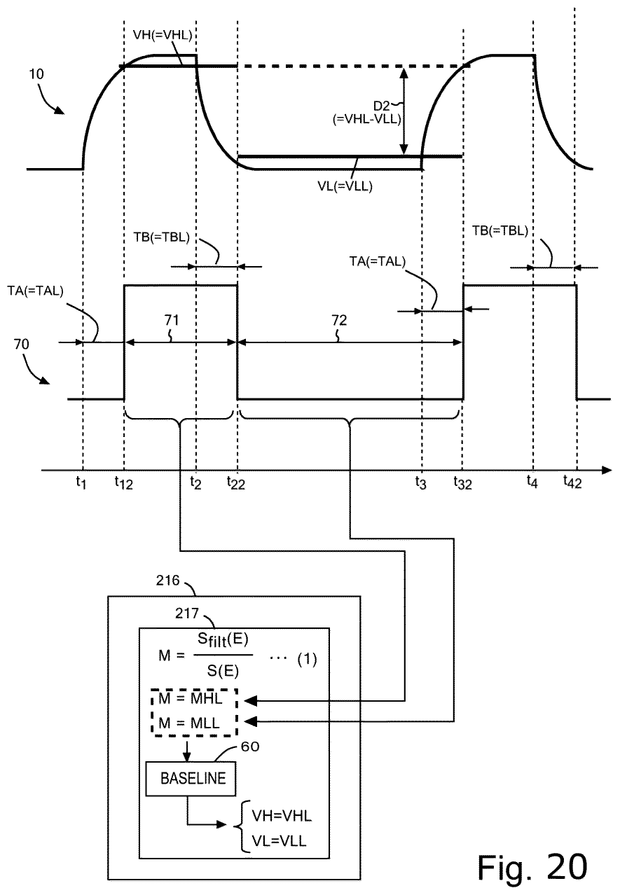

In step ST131, the processor 217 determines signal values Sfilt(E) and S(E) included in equation (1) based on the data received from the DAS 214, and calculates the index value M (=MHL) corresponding to high kV and the index value M (=MLL) corresponding to low kV (see FIG. 19).

FIG. 19 is an explanatory diagram of a method for calculating the index value M (=MHL) corresponding to high kV and the index value M (=MLL) corresponding to low kV.

The processor 217 determines the signal values Sfilt(E) and S(E) in equation (1) based on the data obtained from the reference region 109 (see FIG. 10) during a period 71 in which high kV data is collected, and substitutes the determined signal values Sfilt(E) and S(E) into equation (1) to calculate the index value M (=MHL) corresponding to high kV. Note that in a kV switching scan, when N-number of periods 71 appear during which high kV data is collected, the signal values Sfilt(E) and S(E) may be determined based on all data obtained in the N-number of periods 71, or may be determined based on data obtained in M−(<N) number of periods 71 out of the N-number of periods 71.

Furthermore, the processor 217 determines the signal values Sfilt(E) and S(E) in equation (1) based on the data obtained from the reference region 109 during a period 72 in which low kV data is collected, and substitutes the determined signal values Sfilt(E) and S(E) into equation (1) to calculate the index value M (=MLL) corresponding to low kV. Note that in a kV switching scan, when N-number of periods 72 appear during which low kV data is collected, the signal values Sfilt(E) and S(E) may be determined based on all data obtained in the N-number of periods 72, or may be determined based on data obtained in M−(<N) number of periods 72 out of the N-number of periods 72.

After calculating the index value M (=MHL) corresponding to high kV and the index value M (=MLL) corresponding to low kV, the process proceeds to step ST132.

In step ST132, the processor 217 determines a first effective voltage value corresponding to high kV based on the index value M (=MHL), and determines a second effective voltage value corresponding to low kV based on the index value M (=MLL) (see FIG. 20).

FIG. 20 is an explanatory diagram of a method for determining the effective voltage value.

Based on the baseline 60, the processor 217 determines the first effective voltage value VH (=VHL) corresponding to the high-kV index value M (=MHL) and the second effective voltage value VL (=VLL) corresponding to the low-kV index value M (=MLL). A method for determining the effective voltage value is the same as that described with reference to FIG. 17, and therefore a detailed description thereof will be omitted. The tube voltage waveform 10 in FIG. 20 indicates the effective voltage value VHL and the effective voltage value VLL determined using the baseline 60. After determining the effective voltage value VHL and the effective voltage value VLL, the process proceeds to step ST133.

In step ST133, the processor 217 calculates the second effective voltage difference D2 representing the difference between the first effective voltage value VH (=VHL) corresponding to high kV and the second effective voltage value VL (=VLL) corresponding to low kV. The second effective voltage difference D2 is, as depicted in FIG. 20, D2=VHL−VLL. The second effective voltage difference D2 is stored in the storing device in association with the effective voltage value (VHL, VLL) and the long delay time (TAL, TBL). After calculating the second effective voltage difference D2, the process proceeds to step ST14.

In step ST14, the processor determines whether or not kV switching has been executed with a delay time longer than the reference delay time. Herein, kV switching was executed with a delay time longer than the reference delay time, and thus the process proceeds to step ST16.

In step ST16, the processor determines whether or not kV switching has been executed with a delay time shorter than the reference delay time. Herein, kV switching with the reference delay time and kV switching with a delay time longer than the reference delay time have been executed, but kV switching with a delay time shorter than the reference delay time has not been executed. Therefore, the process proceeds to step ST17.

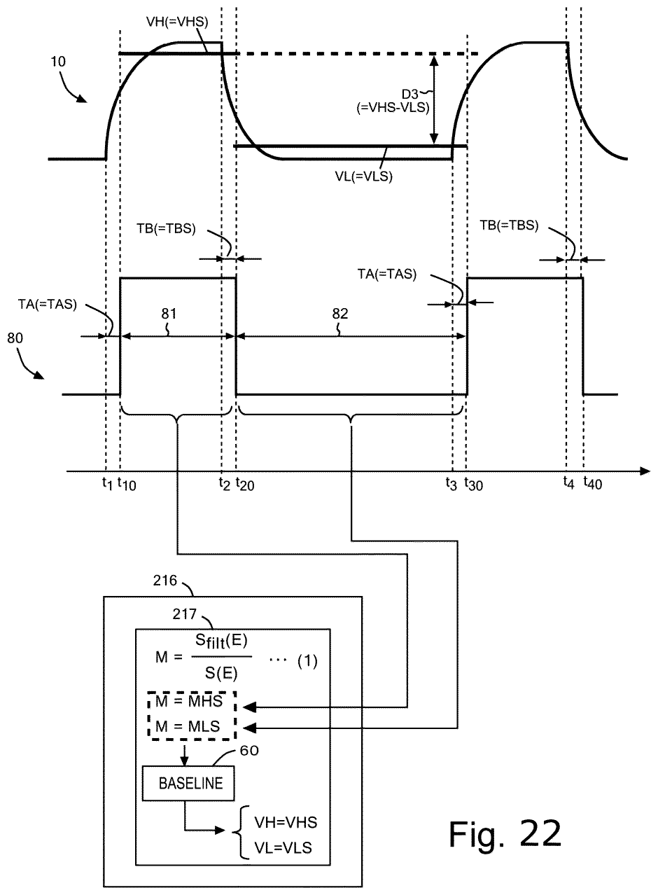

In step ST17, the processor 217 sets the delay time to a time shorter than the reference delay time (see FIG. 21).

FIG. 21 is a diagram depicting an example in which the delay time is set to a time shorter than the reference delay time. FIG. 21 depicts the tube voltage waveform 10, and a rectangular waveform 80, in which the delay time is shorter than the reference delay time, is depicted below the tube voltage waveform 10. Note that for reference, the rectangular waveform 30, in which the delay time is set to the reference delay time, is depicted below the rectangular waveform 80.

In the rectangular waveform 80, the delay time TA is set to a delay time TAS that is shorter than the reference delay time TAr by Δa2. Furthermore, the delay time TB is set to a delay time TBS that is shorter than the reference delay time TBr by Δb2. Note that the short delay time TAS and the short delay time TBS may be the same value or different values. Once the delay time is set to a time (TAS, TBS) shorter than the reference delay time (TAr, TBr), the process returns to step ST12.

In step ST12, in a state in which the delay time (TA, TB) is set to a time (TAS, TBS) shorter than the reference delay time (TAr, TBr), kV switching in which the tube voltage is alternately switched between high kV and low kV is executed, and X-rays are detected by the detector assembly 108. The DAS 214 executes prescribed processing, including sampling processing or the like, on data including the energy information of the detected X-rays to convert the data into digital data, which is then output a computer.

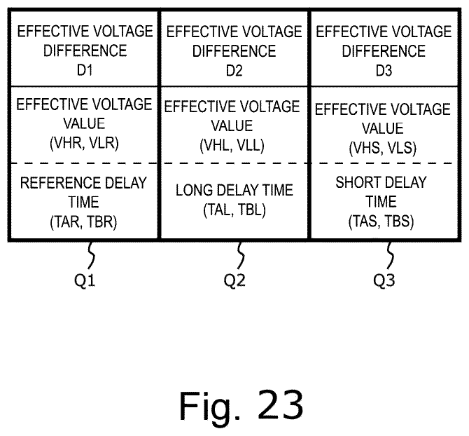

In step ST13, the computer processor 217 calculates a third effective voltage difference D3 (see FIG. 22) based on the data received from the DAS 214. Specifically, the third effective voltage difference D3 is calculated as follows.

In step ST131, the processor 217 determines signal values Sfilt(E) and S(E) included in equation (1) based on the data received from the DAS 214, and calculates the index value M (=MHS) corresponding to high kV and the index value M (=MLS) corresponding to low kV (see FIG. 22).

FIG. 22 is an explanatory diagram of a method for calculating the index value M (=MHS) corresponding to high kV and the index value M (=MLS) corresponding to low kV. The processor 217 determines the signal values Sfilt(E) and S(E) in equation (1) based on the data obtained from the reference region 109 (see FIG. 10) during a period 81 in which high kV data is collected, and substitutes the determined signal values Sfilt(E) and S(E) into equation (1) to calculate the index value M (=MHS) corresponding to high kV. Note that in a kV switching scan, when N-number of periods 81 appear during which high kV data is collected, the signal values Sfilt(E) and S(E) may be determined based on all data obtained in the N-number of periods 81, or may be determined based on data obtained in M−(<N) number of periods 81 out of the N-number of periods 81.

Furthermore, the processor 217 determines the signal values Sfilt(E) and S(E) in equation (1) based on the data obtained from the reference region 109 during a period 82 in which low kV data is collected, and substitutes the determined signal values Sfilt(E) and S(E) into equation (1) to calculate the index value M (=MLS) corresponding to low kV. Note that in a kV switching scan, when N-number of periods 82 appear during which low kV data is collected, the signal values Sfilt(E) and S(E) may be determined based on all data obtained in the N-number of periods 82, or may be determined based on data obtained in M−(<N) number of periods 82 out of the N-number of periods 82.

After calculating the index value M (=MHS) corresponding to high kV and the index value M (=MLS) corresponding to low kV, the process proceeds to step ST132.

In step ST132, based on the baseline 60, the processor 217 determines the first effective voltage value VH (=VHS) corresponding to the high-kV index value M (=MHS) and the second effective voltage value VL (=VLS) corresponding to the low-kV index value M (=MLS). A method for determining the effective voltage value is the same as that described with reference to FIG. 17, and therefore a detailed description thereof will be omitted. The tube voltage waveform 10 in FIG. 22 indicates the effective voltage value VHS and the effective voltage value VLS determined using the baseline 60. After determining the effective voltage value VHS and the effective voltage value VLS, the process proceeds to step ST133.

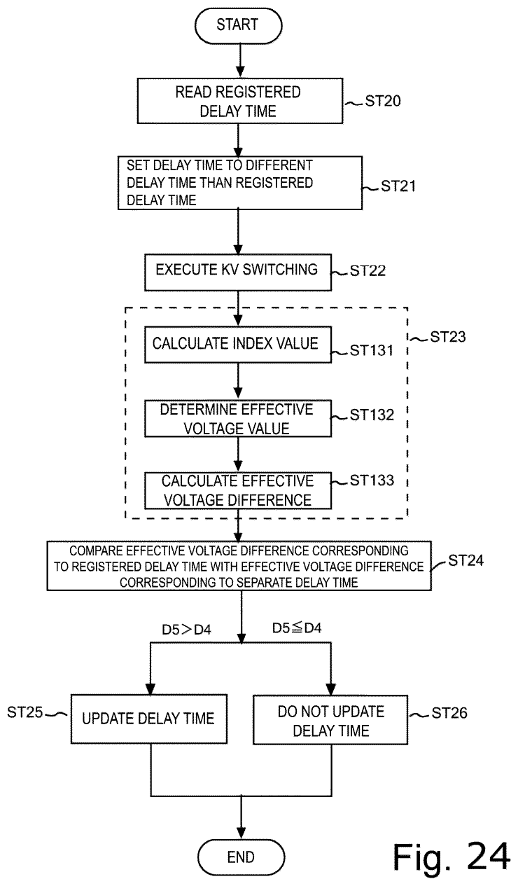

In step ST133, the processor 217 calculates the third effective voltage difference D3 representing the difference between the first effective voltage value VH (=VHS) corresponding to high kV and the second effective voltage value VL (=VLS) corresponding to low kV. The third effective voltage difference D3 is, as depicted in FIG. 22, D3=VHS−VLS. The third effective voltage difference D3 is stored in the storing device in association with the effective voltage value (VHS, VLS) and the short delay time (TAS, TBS). Therefore, the storing device stores data sets Q1, Q2, and Q3, as depicted in FIG. 23.

The data set Q1 includes the first effective voltage difference D1, and effective voltage values (VHr, VLr) and reference delay times (TAr, TBr) associated with the first effective voltage difference D1 (see FIG. 16).

The data set Q2 includes the second effective voltage difference D2, and effective voltage values (VHL, VLL) and long delay times (TAL, TBL) associated with the second effective voltage difference D2 (see FIG. 20).

The data set Q3 includes the third effective voltage difference D3, and effective voltage values (VHS, VLS) and short delay times (TAS, TBS) associated with the third effective voltage difference D3 (see FIG. 22).

After calculating the third effective voltage difference D3, the process proceeds to step ST14.

In step ST14, the processor 217 determines whether or not kV switching has been executed with a delay time longer than the reference delay time. Herein, kV switching was executed with a delay time longer than the reference delay time, and thus the process proceeds to step ST16.

In step ST16, the processor 217 determines whether or not kV switching has been executed with a delay time shorter than the reference delay time. Herein, kV switching was executed with a delay time shorter than the reference delay time, and thus the process proceeds to step ST18.

In step ST18, the processor 217 optimizes the first delay time TA and the second delay time TB based on the first effective voltage difference D1, the second effective voltage difference D2, and the third effective voltage difference D3. Specifically, the processor 217 obtains the first delay time TA and the second delay time TB when the maximum effective voltage difference is obtained based on the first effective voltage difference D1, the second effective voltage difference D2, and the third effective voltage difference D3. An example of a method for obtaining the first delay time TA and the second delay time TB when a maximum effective voltage difference is obtained will be described below.

The processor 217 reads the data sets Q1 to Q3 from the storing device. Next, the processor 217 optimizes the first delay time TA and the second delay time TB based on the data sets Q1 to Q3 such that a maximum effective voltage difference is obtained, and obtains a first optimal delay time TAO representing an optimal value of the first delay time TA and a second optimal delay time TBO representing an optimal value of the second delay time TB. The processor can obtain the first optimal delay time TAO and second optimal delay time TBO based on the effective voltage differences D1 to D3 and the delay times (TAr, TBr), (TAL, TBL), and (TAS, TBS), for example, by adding a certain constraint (e.g., constraint that the effective voltage differences and delay times are expressed by a normal distribution, or the like). Note that the data set including the maximum effective voltage difference may be identified from the data sets Q1 to Q3, and the delay times included in the data set may be identified as optimal delay times (TAO, TBO). For example, among the data sets Q1 to Q3, if the data set including the maximum effective voltage difference is Q2, the long delay times (TAL, TBL) included in the data set Q2 may be set as the optimal delay times (TAO, TBO).

In this manner, the optimal delay time (TAO, TBO), which is a delay time when the maximum effective voltage difference is obtained, can be obtained. The processor registers the optimal delay time (TAO, TBO) in the storing device and ends the flow.

In CT system installation work, the second effective voltage difference D2 (see FIG. 20) corresponding to the delay time (TAL, TBL) longer than the reference delay time (TAr, TBr) and the third effective voltage difference D3 (see FIG. 22) corresponding to the delay time (TAS, TBS) shorter than the reference delay time (TAr, TBr) are calculated in addition to the first effective voltage difference D1 (see FIG. 16) corresponding to the reference delay time (TAr, TBr). Furthermore, based on these effective voltage differences D1, D2, and D3, the optimal delay time (TAO, TBO), which is the delay time when the maximum effective voltage difference is obtained, can be obtained. Therefore, when actually imaging a subject using a CT examination, projection data corresponding to high kV and projection data corresponding to low kV can be acquired based on the optimal delay time (TAO, TBO), which is the optimized delay time obtained in the flow above. Therefore, projection data corresponding to high kV and projection data corresponding to low kV can be acquired with the delay time when the maximum effective voltage difference is obtained, such that a high-quality CT image can be reconstructed.

Note that in the flow of FIG. 13, kV switching is executed for the reference delay time (TAr, TBr), the long delay time (TAL, TBL), and the short delay time (TAS, TBS). In other words, kV switching is executed for three different delay times. However, two delay times may be set and kV switching may be executed for the two delay times, or four or more delay times may be set and kV switching may be executed for the four or more delay times.

Next, an example will be described in which an optimal delay time is obtained when a user of the CT system periodically calibrates the CT system after the CT system installed in a facility actually starts to operate.

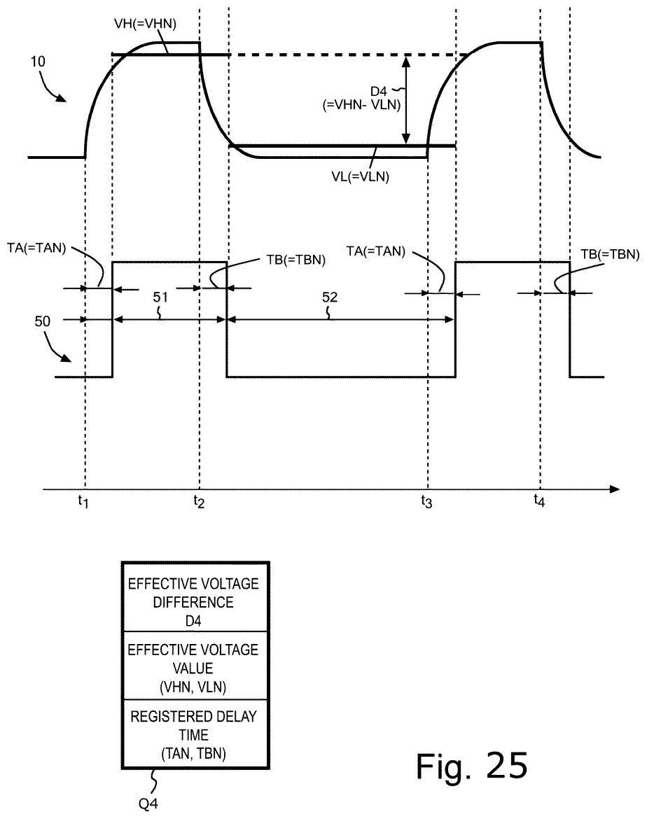

FIG. 24 is a flowchart of a method for obtaining an optimal delay time when a user periodically executes calibration. In step ST20, the processor 217 accesses the storing device and reads the latest value of a registered delay time. FIG. 25 is an explanatory diagram of the latest value of a registered delay time.

The upper part of FIG. 25 depicts the tube voltage waveform 10, and the lower part depicts a rectangular waveform 50. The rectangular waveform 50 represents a period 51 corresponding to a high kV and a period 52 corresponding to a low kV.

The rectangular waveform 50 indicates a first registered delay time TAN and a second registered delay time TBN. The first registered delay time TAN represents a delay time registered as the latest value of the first delay time TA, and the second registered delay time TBN represents a delay time registered as the latest value of the second delay time TB. Note that the delay time TAN and the delay time TBN may be the same value or different values. Furthermore, an effective voltage value VH (=VHN) and an effective voltage value VL (=VLN) are effective voltages values obtained for the periods 51 and 52, respectively. The fourth effective voltage difference D4 represents the difference between the effective voltage value VH (=VHN) and the effective voltage value VL (=VLN). The registered delay times (TAN, TBN), effective voltage values (VHN, VLN), and fourth effective voltage difference D4 are associated with one another and stored in the storing device as a data set Q4, as depicted at the bottom of FIG. 25. The processor 217 accesses the storing device and reads the first registered delay time TAN and the second registered delay time TBN from the data set Q4. When the processor 217 reads the registered delay times (TAN, TBN), the process proceeds to step ST21.

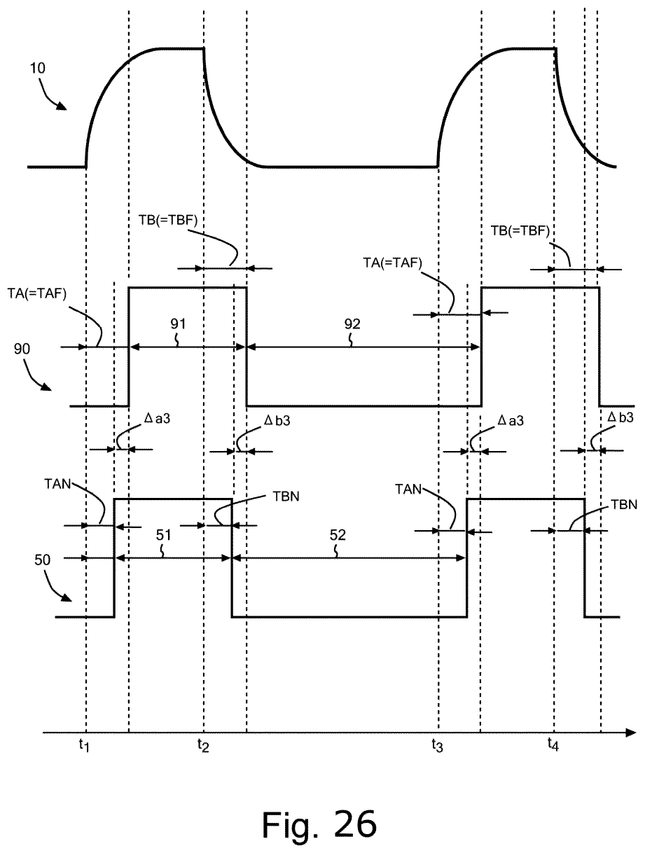

In step ST21, the processor 217 sets the delay time to a delay time different from the registered delay time (TAN, TBN) (see FIG. 26).

FIG. 26 is a diagram depicting the delay time set to a time different from the registered delay time (TAN, TBN).

FIG. 26 depicts the tube voltage waveform 10, and depicts a rectangular waveform 90 below the tube voltage waveform 10.

The rectangular waveform 90 represents a period 91 corresponding to a high kV and a period 92 corresponding to a low kV. The rectangular waveform 90 is provided with a first separate delay time TAF different from the first registered delay time TAN and a second separate delay time TBF different from the second registered delay time TBN.

In the rectangular waveform 90, the first separate delay time TAF is set to a delay time that is longer than the first registered delay time TAN by Δa3. Furthermore, the second separate delay time TBF is set to a delay time that is longer than the second registered delay time TBN by Δb3. Note that in FIG. 26, the separate delay time (TAF, TBF) is a delay time longer than the registered delay time (TAN, TBN), but may also be a delay time shorter than the registered delay time (TAN, TBN). After setting the separate delay time (TAF, TBF), the process proceeds to step ST22.

In step ST22, in a state in which the delay time is set to the separate delay time (TAF, TBF), kV switching is executed, in which the tube voltage is alternately switched between high kV and low kV, and X-rays are detected by the detector assembly 108. As an example, the high kV may be 140 kV and the low kV may be 80 kV. The DAS 214 executes prescribed processing, including sampling processing or the like, on data including the energy information of the detected X-rays to convert the data into digital data, which is then output a computer.

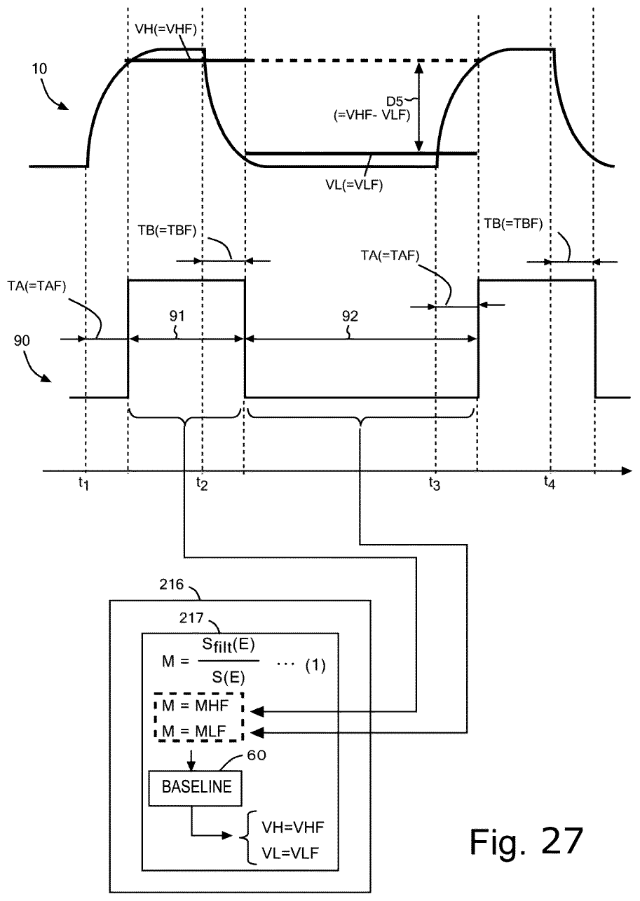

In step ST23, the computer processor 217 calculates a fifth effective voltage difference D5 (see FIG. 27) based on the data received from the DAS 214.

FIG. 27 is an explanatory diagram of a method for calculating the fifth effective voltage difference D5. Note that step ST23 is the same as step ST13 depicted in FIG. 13, and thus will be briefly described below.

In step ST131, the processor 217 determines signal values Sfilt(E) and S(E) included in equation (1) based on the data received from the DAS 214, and calculates an index value M (=MHF) corresponding to high kV and an index value M (=MLF) corresponding to low kV. Note that in a kV switching scan, when N-number of periods 91 appear during which high kV data is collected, the signal values Sfilt(E) and S(E) may be determined based on all data obtained in the N-number of periods 91, or may be determined based on data obtained in M−(<N) number of periods 91 out of the N-number of periods 91. Note that in a kV switching scan, when N-number of periods 92 appear during which low kV data is collected, the signal values Sfilt(E) and S(E) may be determined based on all data obtained in the N-number of periods 92, or may be determined based on data obtained in M−(<N) number of periods 92 out of the N-number of periods 92.

After calculating the index value M (=MHF) corresponding to high kV and the index value M (=MLF) corresponding to low kV, the process proceeds to step ST132.

In step ST132, based on the baseline 60, the processor 217 determines the effective voltage value VH (=VHF) corresponding to the high-kV index value M (=MHF) and the effective voltage value VL (=VLF) corresponding to the low-kV index value M (=MLF). After determining the effective voltage value VHF and the effective voltage value VLF, the process proceeds to step ST133.

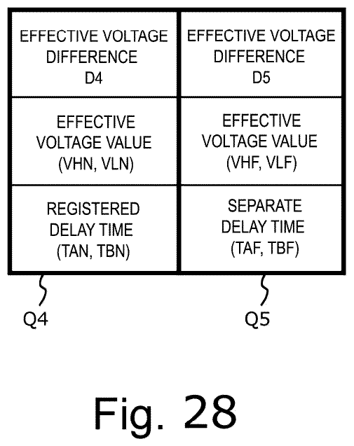

In step ST133, the processor 217 calculates the fifth effective voltage difference D5 representing the difference between the effective voltage value VH (=VHF) corresponding to high kV and the effective voltage value VL (=VLF) corresponding to low kV. The fifth effective voltage difference D5 is, as depicted in FIG. 27, D5=VHF−VLF. The fifth effective voltage difference D5, the effective voltage value (VHF, VLF) and the delay time (TAF, TBF) are stored in the storing device as one data set. FIG. 28 depicts the data sets Q4 and Q5 stored in the storing device. The data set Q4 is a data set including the registered delay time (TAN, TBN), effective voltage value (VHN, VLN), and the fourth effective voltage difference D4 described above with reference to FIG. 25. On the other hand, the data set Q5 is a data set including the separate delay time (TAF, TBF) set in step ST21 of the flow of FIG. 24, and the effective voltage value (VHF, VLF) and effective voltage difference D5 obtained in step ST23. After storing the data set Q5, the process proceeds to step ST24.

In step ST24, the processor 217 compares the fourth effective voltage difference D4 corresponding to the registered delay times (TAN, TBN) of the data set Q4 with the fifth effective voltage difference D5 corresponding to the separate delay times (TAF, TBF) of the data set Q5. If D5>D4, this means that the delay time (TAF, TBF) set in step ST21 can obtain a larger effective voltage difference than the registered delay time (TAN, TBN). Therefore, when D5>D4, the delay time (TAF, TBF) set in step ST21 is considered to be able to improve the image quality more than the registered delay time (TAN, TBN). Thus, when D5>D4, the process proceeds to step ST25, where the delay time is updated to the separate delay time (TAF, TBF) corresponding to the fifth effective voltage difference D5, and then the flow ends.

On the other hand, if D5≤D4, this means that the delay time (TAF, TBF) set in step ST21 has the same effective voltage difference as or a smaller effective voltage difference than the registered delay time (TAN, TBN). Therefore, when D5≤D4, the delay time (TAF, TBF) set in step ST21 is considered to be able to improve the image quality more than by using the registered delay time (TAN, TBN). Therefore, when D5≤D4, the process proceeds to step ST26, where the delay time is not updated and the registered delay time (TAN, TBN) is maintained, and then the flow ends.

In the flow depicted in FIG. 24, one calibration scan is executed for the delay time (TAF, TBF) set in step ST21, and whether or not to update the delay time is determined based on the data obtained by the one calibration scan. Therefore, the flow depicted in FIG. 24 executes only one calibration scan, and is therefore suitable for calibration in which finishing the calibration in as short a time as possible is a priority. For example, a user may be recommended to execute a daily calibration approximately once a day. However, daily calibration is a task that must be executed every day, in principle, in the midst of the busy daily work of the user, and thus from the standpoint of the user, daily calibration is a task to be completed as quickly as possible. Therefore, by incorporating the flow depicted in FIG. 24 into the daily calibration, the user can complete the work of updating the delay time with a single calibration scan, thereby shortening the time required for calibration.

Note that the present invention can be applied not only to a full scan but also to a half scan, so long as the scan is executed while switching the tube voltage.

Note that in the present embodiment, an example is described in which the CT system 100 is used as a medical system. However, the present invention is not limited to the CT system 100 and can be applied to a system other than the CT system 100 (e.g., PET-CT system), so long as the medical system irradiates an X-ray source onto the subject.

1. A medical system, comprising:
an X-ray tube configured to be able to switch a tube voltage applied to the X-ray tube between a first tube voltage and a second tube voltage;
a detector assembly for detecting X-rays irradiated from the X-ray tube, the detector assembly including a reference region for detecting X-rays and a filter, the reference region including a first sub-region for detecting X-rays that have passed through the filter and a second sub-region for detecting X-rays that have not passed through the filter; and
one or a plurality of processors for executing the following:
setting a first delay time from switching the tube voltage to the first tube voltage to starting data collection, and a second delay time from switching the tube voltage to the second tube voltage to starting data collection;
determining a first effective voltage value of the first tube voltage based on energy information of X-rays detected in the first sub-region of the detector assembly and energy information of X-rays detected in the second sub-region of the detector assembly, when the first tube voltage is applied to the X-ray tube;
determining a second effective voltage value of the second tube voltage based on energy information of X-rays detected in the first sub-region of the detector assembly and energy information of X-rays detected in the second sub-region of the detector assembly, when the second tube voltage is applied to the X-ray tube; and
calculating an effective voltage difference representing the difference between the first effective voltage value and the second effective voltage value, wherein
the one or more processors execute, a plurality of times,
setting the first delay time and the second delay time, determining the first effective voltage value, determining the second effective voltage value, and calculating the effective voltage difference, to acquire a plurality of effective voltage differences, and obtain the first delay time and the second delay time when a maximum effective voltage difference is obtained based on the plurality of effective voltage differences.

2. The medical system according to claim 1, wherein a first reference delay time representing a reference value of the first delay time and a second reference delay time representing a reference value of the second delay time are stored in a storing device included in the medical system or a storing device accessible by the medical system, and
when a first calibration is executed on the medical system, the one or plurality of processors execute the following:
calculating a first effective voltage difference representing a difference between the first effective voltage value and the second effective voltage value based on data obtained when the first delay time is set as the first reference delay time and the second delay time is set as the second reference delay time;
calculating a second effective voltage difference representing a difference between the first effective voltage value and the second effective voltage value based on data obtained when the first delay time is set to a delay time longer than the first reference delay time and the second delay time is set to a delay time longer than the second reference delay time;
calculating a third effective voltage difference representing a difference between the first effective voltage value and the second effective voltage value based on data obtained when the first delay time is set to a delay time shorter than the first reference delay time and the second delay time is set to a delay time shorter than the second reference delay time; and
obtaining the first delay time and the second delay time when a maximum effective voltage difference is obtained based on the first effective voltage difference, the second effective voltage difference, and the third effective voltage difference.

3. The medical system according to claim 2, wherein the first calibration is executed at the time of installation work, maintenance work, or part replacement of the medical system.

4. The medical system according to claim 2, wherein the first delay time and the second delay time are the same value or different values.

5. The medical system according to claim 1, wherein a first registered delay time representing a delay time registered as a latest value of the first delay time, a second registered delay time representing a delay time registered as a latest value of the second delay time, and a fourth effective voltage difference corresponding to the first registered delay time and the second registered delay time are stored in a storing device included in the medical system or a storing device accessible by the medical system, and
when a second calibration is executed on the medical system, the one or plurality of processors execute the following:
setting the first delay time as a first separate delay time different from the first registered delay time, and setting the second delay time as a second separate delay time different from the second registered delay time;
calculating a fifth effective voltage difference representing a difference between the first effective voltage value and the second effective voltage value based on data obtained when the first delay time is set as the first separate delay time and the second delay time is set as the second separate delay time; and
updating the first delay time to the first separate delay time and updating the second delay time to the second separate delay time when the fifth effective voltage difference is greater than the fourth effective voltage difference.

6. The medical system according to claim 5, wherein the second calibration is periodically executed by a user.

7. The medical system according to claim 1, wherein determining the first effective voltage value includes:
determining the first effective voltage value based on a signal value including energy information of X-rays detected in the first sub-region and a signal value including energy information of X-rays detected in the second sub-region, and
determining the second effective voltage value includes:
determining the second effective voltage value based on a signal value including energy information of X-rays detected in the first sub-region and a signal value including energy information of X-rays detected in the second sub-region.

8. The medical system according to claim 7, wherein determining the first effective voltage value includes:
calculating a first index value based on a signal value including energy information of the X-rays detected in the first sub-region and a signal value including energy information of the X-rays detected in the second sub-region; and
determining the first effective voltage value based on the first index value, and
determining the second effective voltage value includes:
calculating a second index value based on a signal value including energy information of the X-rays detected in the first sub-region and a signal value including energy information of the X-rays detected in the second sub-region; and
determining the second effective voltage value based on the second index value.

9. The medical system according to claim 8, wherein the first index value and the second index value are calculated by a ratio between a signal value including energy information of X-rays detected in the first sub-region and a signal value including energy information of X-rays detected in the second sub-region.

10. The medical system according to claim 9, wherein the one or more processors
each calculate the first effective voltage value and the second effective voltage value based on a baseline that associates a tube voltage with an index value.

11. The medical system according to claim 10, wherein the baseline is a line created based on a plurality of reference tube voltages and a plurality of index values corresponding to the plurality of reference tube voltages, and
each of the plurality of index values is a ratio between a signal value including energy information of X-rays detected in the first sub-region and a signal value including energy information of X-rays detected in the second sub-region.

12. A non-transitory storing medium in which is stored an instruction executed by one or a plurality of processors of a medical system including:
an X-ray tube configured to be able to switch a tube voltage applied to the X-ray tube between a first tube voltage and a second tube voltage;
a detector assembly for detecting X-rays irradiated from the X-ray tube, the detector assembly including a reference region for detecting X-rays and a filter, the reference region including a first sub-region for detecting X-rays that have passed through the filter and a second sub-region for detecting X-rays that have not passed through the filter; and
one or a plurality of processors, wherein
the instruction, upon being executed by the one or plurality of processors, causes the one or plurality of processors to execute the following:
setting a first delay time from switching the tube voltage to the first tube voltage to starting data collection, and a second delay time from switching the tube voltage to the second tube voltage to starting data collection;
determining a first effective voltage value of the first tube voltage based on energy information of X-rays detected in the first sub-region of the detector assembly and energy information of X-rays detected in the second sub-region of the detector assembly, when the first tube voltage is applied to the X-ray tube;
determining a second effective voltage value of the second tube voltage based on energy information of X-rays detected in the first sub-region of the detector assembly and energy information of X-rays detected in the second sub-region of the detector assembly, when the second tube voltage is applied to the X-ray tube; and
calculating an effective voltage difference representing the difference between the first effective voltage value and the second effective voltage value, and
the instruction, upon being executed by the one or plurality of processors, causes the one or plurality of processors to further execute, a plurality of times,
setting the first delay time and the second delay time, determining the first effective voltage value, determining the second effective voltage value, and calculating the effective voltage difference, to acquire a plurality of effective voltage differences, and obtain the first delay time and the second delay time when a maximum effective voltage difference is obtained based on the plurality of effective voltage differences.
